SEQUENCE LISTING (4 sequences)

SEQ ID NO: 1 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 2 - DNA, 6070 bp, Homo sapiens
  source 1..6070: mol_type = genomic DNA
gtagtaggtctttggcattaggagcttgagcccagacggccctagcagggaccccagcgcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggagctcgtgggacagtcacctcatggaattggagctcgtggaacagttacctctgcctcagaaaacaaggatgaattaagtttttttttaaaaaagaaacatttggtaaggggaattgaggacactgatatgggtcttgataaatggcttcctggcaatagtcaaattgtgtgaaaggtacttcaaatccttgaagatttaccacttgtgttttgcaagccagattttcctgaaaacccttgccatgtgctagtaattggaaaggcagctctaaatgtcaatcagcctagttgatcagcttattgtctagtgaaactcgttaatttgtagtgttggagaagaactgaaatcatacttcttagggttatgattaagtaatgataactggaaacttcagcggtttatataagcttgtattcctttttctctcctctccccatgatgtttagaaacacaactatattgtttgctaagcattccaactatctcatttccaagcaagtattagaataccacaggaaccacaagactgcacatcaaaatatgccccattcaacatctagtgagcagtcaggaaagagaacttccagatcctggaaatcagggttagtattgtccaggtctaccaaaaatctcaatatttcagataatcacaatacatcccttacctgggaaagggctgttataatctttcacaggggacaggatggttcccttgatgaagaagttgatatgccttttcccaactccagaaagtgacaagctcacagacctttgaactagagtttagctggaaaagtatgttagtgcaaattgtcacaggacagcccttctttccacagaagctccaggtagagggtgtgtaagtagataggccatgggcactgtgggtagacacacatgaagtccaagcatttagatgtataggttgatggtggtatgttttcaggctagatgtatgtacttcatgctgtctacactaagagagaatgagagacacactgaagaagcaccaatcatgaattagttttatatgcttctgttttataattttgtgaagcaaaattttttctctaggaaatatttattttaataatgtttcaaacatatataacaatgctgtattttaaaagaatgattatgaattacatttgtataaaataatttttatatttgaaatattgactttttatggcactagtatttctatgaaatattatgttaaaactgggacaggggagaacctagggtgatattaaccaggggccatgaatcaccttttggtctggagggaagccttggggctgatgcagttgttgcccacagctgtatgattcccagccagcacagcctcttagatgcagttctgaagaagatggtaccaccagtctgactgtttccatcaagggtacactgccttctcaactccaaactgactcttaagaagactgcattatatttattactgtaagaaaatatcacttgtcaataaaatccatacatttgtgtgaaa

SEQ ID NO: 3 - protein, 381 aa, Homo sapiens
MAESHLLQWLLLLLPTLCGPGTAAWTTSSLACAQGPEFWCQSLEQALQCRALGHCLQEVWGHVGADDLCQECEDIVHILNKMAKEAIFQDTMRKFLEQECNVLPLKLLMPQCNQVLDDYFPLVIDYFQNQTDSNGICMHLGLCKSRQPEPEQEPGMSDPLPKPLRDPLPDPLLDKLVLPVLPGALQARPGPHTQDLSEQQFPIPLPYCWLCRALIKRIQAMIPKGALAVAVAQVCRVVPLVAGGICQCLAERYSVILLDTLLGRMLPQLVCRLVLRCSMDDSAGPRSPTGEWLPRDSECHLCMSVTTQAGNSSEQAIPQAMLQACVGSWLDREKCKQFVEQHTPQLLTLVPRGWDAHTTCQALGVCGTMSSPLQCIHSPDL

SEQ ID NO: 4 - DNA, 3550 bp, Homo sapiens
  source 1..3550: mol_type = genomic DNA
aggctgcagaggtgccatggctgagtcacacctgctgcagtggctgctgctgctgctgcccacgctctgtggcccaggcactgctgcctggaccacctcatccttggcctgtgcccagggccctgagttctggtgccaaagcctggagcaagcattgcagtgcagagccctagggcattgcctacaggaagtctggggacatgtgggagccgatgacctatgccaagagtgtgaggacatcgtccacatccttaacaagatggccaaggaggccattttccaggacacgatgaggaagttcctggagcaggagtgcaacgtcctccccttgaagctgctcatgccccagtgcaaccaagtgcttgacgactacttccccctggtcatcgactacttccagaaccagactgactcaaacggcatctgtatgcacctgggcctgtgcaaatcccggcagccagagccagagcaggagccagggatgtcagaccccctgcccaaacctctgcgggaccctctgccagaccctctgctggacaagctcgtcctccctgtgctgcccggggccctccaggcgaggcctgggcctcacacacaggatctctccgagcagcaattccccattcctctcccctattgctggctctgcagggctctgatcaagcggatccaagccatgattcccaagggtgcgctagctgtggcagtggcccaggtgtgccgcgtggtacctctggtggcgggcggcatctgccagtgcctggctgagcgctactccgtcatcctgctcgacacgctgctgggccgcatgctgccccagctggtctgccgcctcgtcctccggtgctccatggatgacagcgctggcccaaggtcgccgacaggagaatggctgccgcgagactctgagtgccacctctgcatgtccgtgaccacccaggccgggaacagcagcgagcaggccataccacaggcaatgctccaggcctgtgttggctcctggctggacagggaaaagtgcaagcaatttgtggagcagcacacgccccagctgctgaccctggtgcccaggggctgggatgcccacaccacctgccaggccctcggggtgtgtgggaccatgtccagccctctccagtgtatccacagccccgacctttgatgagaactcagctgtccagctgcaaaggaaaagccaagtgagacgggctctgggaccatggtgaccaggctcttcccctgctccctggccctcgccagctgccaggctgaaaagaagcctcagctcccacaccgccctcctcaccgcccttcctcggcagtcacttccactggtggaccacgggcccccagccctgtgtcggccttgtctgtctcagctcaaccacagtctgacaccagagcccacttccatcctctctggtgtgaggcacagcgagggcagcatctggaggagctctgcagcctccacacctaccacgacctcccagggctgggctcaggaaaaaccagccactgctttacaggacagggggttgaagctgagccccgcctcacacccacccccatgcactcaaagattggattttacagctacttgcaattcaaaattcagaagaataaaaaatgggaacatacagaactctaaaagatagacatcagaaattgttaagttaagctttttcaaaaaatcagcaattccccagcgtagtcaagggtggacactgcacgctctggcatgatgggatggcgaccgggcaagctttcttcctcgagatgctctgctgcttgagagctattgctttgttaagatataaaaaggggtttctttttgtctttctgtaaggtggacttccagcttttgattgaaagtcctagggtgattctatttctgctgtgatttatctgctgaaagctcagctggggttgtgcaagctagggacccattcctgtgtaatacaatgtctgcaccaatgctaataaagtcctattctcttttatgagaaagaaaaagacaccgtcctttaaagtgctgcagtatggccagacgtggtggctcacacctgcaatcccagcaccttaggaggccgaggcaggaggatccttgaggtcaggagttcgagaccagcctcgccaacatggtgaaaccccatttctactaaaaatacaaaaaattagccaagtgtggtggcatatgcctgtaatcccaactactcagaaggccgaggcaggagaattacttgaacgcaggagaatcactgcagcccaggaggcagaggttgcagtgagccgagattgcaccactgcactccagcctgggtgacagagcaagactccatctcagtaaataaataaataaataaaaagcgctgcagtagctgtggcctcaccctgaagtcagcgggcccaggcctacctcactctctcccttggcagagaagcagacgtccatagctcctctccctcacaagcgctcccagcctgccctccagctgctgctctcccctcccagtctctactcactgggatgaggttaggtcatgaggacaccaaaaacctaaaaataaacaaaaagccaaacaagccttagcttttcttaaagactgaaatgcctggaagtgtccctttatttataaaataacttttgtcatatttcttatacatgtttcttgtaagaaattcagaaactacagacaaagagagtggaaattacccactgtcaggcctctgagcccaagctaagccatcatatcccctgtgccctgcacgtatacacccagatggcctgaagcaactgaagatccacaaaagaagtgaaaatagccagttcctgccttaactgatgacattccaccattgtgatttgttcctgccccaccctaactgatcaattgaccttgtgacaatacaccttccccacccttgagaaggtgctttgtaatattctccccacccaccccacgcccgcacccccgcacccttaagaaggtattttgtaatattctctccgccattgagaatgtgctttgtaagatccaccccctgcccacaaaaaattgctcctaactccaccgcctatcccaaacctacaagaactaatgataatcccaccaccctttgctgactctttttggactcagcccacctgcacccaggtgattaaaaagctttattgttcacacaaagcctgtttggtagtctcttcacagggaagcatgtgacacccacaatcccacctagcccaggagagagctacggcagggtgtgtgttttgacactgagcttggggctttttccatcttctccccacagcctctggctccacacctccaccgttcaagcgccagaaagagctgtctatgcagcctgctcttgggcctggggatgagacacacaattcattggctcctggattttaagtagacatttgtaaatctatagctaactactgtccttaaagccattgtttccattacaaaatccaactctctgagagaaaagggtgttttaaatttaaaaaaataaaaacaaaaaagtttgattgagacaa

(19) United States
(12) Patent Application Publication
(10) Pub. No.: US 2025/0082783 A1
(43) Pub. Date: Mar. 13, 2025

(54) COMPOSITIONS AND METHODS FOR PREVENTION AND TREATMENT OF GENETIC DISEASE

(71) Applicant: THE CHILDREN'S MEDICAL CENTER CORPORATION, Boston, MA (US)

(72) Inventor: Dario O. Fauza, Boston, MA (US)

(21) Appl. No.: 18/890,368

(22) Filed: Sep. 19, 2024

Related U.S. Application Data
(63) Continuation of application No. PCT/US2023/015766, filed on Mar. 21, 2023.
(60) Provisional application No. 63/322,576, filed on Mar. 22, 2022.

(51) Int. Cl. A61K 48/00; A61K 38/17; A61P 11/00

(52) U.S. Cl. CPC A61K 48/0058; A61K 38/1709; A61K 38/395; A61K 48/0033; A61P 11/00

(57) ABSTRACT

Methods and compositions for the transamniotic delivery of mRNA encoding cystic fibrosis transmembrane conductance regulator protein or surfactant protein B to a fetus for the prenatal treatment of cystic fibrosis or a surfactant protein B deficiency.

### CROSS-REFERENCE TO RELATED APPLICATION

This application is a continuation of International Application No. PCT/US2023/015766, filed on Mar. 21, 2023, which claims priority to and benefit of U.S. Provisional Application No. 63/322,576, filed on Mar. 22, 2022, the entire contents of which are incorporated by reference.

### SEQUENCE LISTING

This application contains a Sequence Listing which has been filed electronically in XML format and is hereby incorporated by reference in its entirety. The Sequence Listing XML, created on Nov. 18, 2024, is named 167705-031202USCON_SL.xml and is 15,999 bytes in size.

### BACKGROUND OF THE INVENTION

Therapies based on exogenous mRNA administration have emerged as a powerful novel strategy for the actual or potential treatment of an assortment of diseases, including congenital pathologies. Accordingly, there is a need for an alternative for fetal mRNA delivery, such as the simpler and minimally invasive transamniotic route.

### SUMMARY OF THE INVENTION

In one aspect, the disclosure features a recombinant polypeptide method for treating cystic fibrosis in a developing fetus. The method involves administering to the amniotic fluid surrounding a developing fetus an mRNA molecule encoding a cystic fibrosis transmembrane conductance regulator (CFTR) polypeptide, thereby treating cystic fibrosis in the developing fetus.

In another aspect, the disclosure features a method for treating a disease or disorder associated with a surfactant protein B (SPB) deficiency in a developing fetus, the method including: administering to the amniotic fluid surrounding a developing fetus an mRNA molecule encoding an SPB polypeptide, thereby treating the disease or disorder associated with SPB deficiency in the developing fetus.

In another aspect, the disclosure features a kit suitable for use in carrying out the method of any aspect of the disclosure delineated herein, or embodiments thereof, where the kit includes the mRNA of any aspect of the disclosure delineated herein, or embodiments thereof.

In another aspect, the disclosure features a method for delivering one or more mRNA molecules to the bloodstream of a developing fetus, the method including: administering to the amniotic fluid surrounding a developing fetus one or more mRNA molecules encoding for polypeptides or proteins either missing or defective in a developing fetus.

In any aspect of the disclosure delineated herein, or embodiments thereof, the mRNA is translated in the lungs and/or intestines of the fetus to produce the CFTR polypeptide in lung cells of the fetus.

In any aspect of the disclosure delineated herein, or embodiments thereof, the method includes reducing the incidence of meconium ileus in the fetus or the child developed from the fetus.

In the method of any aspect of the disclosure delineated herein, or embodiments thereof, the disease or disorder includes pulmonary alveolar proteinosis. In some embodiments, the mRNA is translated in small bowel cells of the fetus to produce the SPB polypeptide in small bowel cells of the fetus. In some embodiments, the mRNA is translated in airway and/or pulmonary cells of the fetus to produce the SPB polypeptide in airway and/or pulmonary cells of the fetus. In some embodiments, the method is associated with an increase in levels of phosphatidylcholine in the amniotic fluid.

In any aspect of the disclosure delineated herein, or embodiments thereof, the mRNA is administered using a lipid nanoparticle or a lipopolyplex. In some embodiments, the lipopolyplex contains a cationic polymer. In some embodiments, the method is associated with increased life expectancy for the child developed from the fetus.

In any of the above aspects, the mRNA is codon-optimized. In any of the above aspects, the polypeptide is a human polypeptide. In any of the above aspects, the fetus is a mammalian fetus. In some aspects, the fetus is a human fetus.

In any of the above aspects, the mRNA is administered prior to 10 weeks of pregnancy. In any of the above aspects, the mRNA is administered prior to 5 weeks of pregnancy. In any of the above aspects, the mRNA is administered after 10 weeks of pregnancy.

In any of the above aspects, the administration is associated with the presence of and/or an increase in levels of the mRNA in a tissue of the fetus. In some embodiments, the tissue includes liver, stomach, intestines, pancreas, spleen, thymus, lymph nodes, brain, meninges, heart, blood vessels, lungs, airways, kidneys, ureters, urethra, ovaries, testicles, genitalia, skin, skin annexes, muscle, bone, cartilage, bone marrow, eyes, ears, mouth, nose, pharynx, larynx, vitreous body, cerebrospinal fluid, and/or peripheral blood. In some embodiments, the administration is associated with the presence of and/or an increase in levels of the mRNA in amniotic fluid, amnion, chorion, umbilical cord, and/or placenta associated with the fetus. In any of the above aspects, the mRNA is translated in an organ and/or tissue of the developing fetus, thereby treating the developing fetus for a disease or disorder associated with the polypeptide. In the method of any aspect of the disclosure delineated herein, or embodiments thereof, the tissue contains liver, stomach, intestines, lungs, and/or blood. In the method of any aspect of the disclosure delineated herein, the polypeptide is surfactant protein A. In the method of any aspect of the disclosure delineated herein, the polypeptide is surfactant protein B. In the method of any aspect of the disclosure delineated herein, the polypeptide is surfactant protein C. In the method of any aspect of the disclosure delineated herein, the polypeptide is surfactant protein D.

In the method of any aspect of the disclosure delineated herein, the disease or disorder is prematurity or congenital surfactant protein B deficiency.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof. In some embodiments, the agent is an mRNA molecule. In some instances, the mRNA molecule encodes cystic fibrosis transmembrane conductance regulator (CFTR) protein or surfactant protein B (SPB).

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels. “

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “cystic fibrosis transmembrane conductance regulator (CFTR) polypeptide” is meant a polypeptide or fragment thereof having at least 85% amino acid sequence identity to NCBI reference sequence NO.: NP_000483.3 or a fragment thereof capable of mediating transport of ions across a membrane. An exemplary CFTR polypeptide sequence is provided below:

### >NP 000483.3

| (SEQ ID NO: 1) | |
|---|---|
| 1 | mqrsplekas vvsklffswt rpilrkgyrq rlelsdiyqi psvdsadnls eklerewdre |
| 61 | laskknpkli nalrrcffwr fmfygiflyl gevtkavqpl llgriiasyd pdnkeersia |
| 121 | iylgiglcll fivrtlllhp aifglhhigm qmriamfsli ykktlklssr vldkisigql |
| 181 | vsllsnnlnk fdeglalahf vwiaplqval lmgliwellq asafcglgfl ivlalfqagl |
| 241 | grmmmkyrdq ragkiserlv itsemieniq svkaycweea mekmienlrq telkltrkaa |
| 301 | yvryfnssaf fisgffvvfl svlpyalikg iilrkiftti sfcivlrmav trqfpwavqt |
| 361 | wydslgaink iqdflqkqey ktleynlttt evvmenvtaf weegfgelfe kakqnnnnrk |
| 421 | tsngddslff snfsllgtpv lkdinfkier gqllavagst gagktsllmv imgelepseg |
| 481 | kikhsgrisf csqfswimpg tikeniifgv sydeyryrsv ikacqleedi skfaekdniv |
| 541 | lgeggitlsg gqrarislar avykdadlyl ldspfgyldv ltekeifesc vcklmanktr |
| 601 | ilvtskmehl kkadkililh egssyfygtf selqnlqpdf ssklmgcdsf dqfsaerrns |
| 661 | iltetlhrfs legdapvswt etkkqsfkqt gefgekrkns ilnpinsirk fsivqktplq |
| 721 | mngieedsde plerrlslvp dseqgeailp risvistgpt lqarrrqsvl nlmthsvnqg |
| 781 | qnihrkttas trkvslapqa nlteldiysr rlsqetglei seeineedlk ecffddmesi |
| 841 | pavttwntyl ryitvhksli fvliwclvif laevaaslvv lwllgntplq dkgnsthsrn |
| 901 | nsyaviitst ssyyvfyiyv gvadtllamg ffrglplvht litvskilhh kmlhsvlqap |
| 961 | mstlntlkag gilnrfskdi ailddllplt ifdfiqllli vigaiavvav lqpyifvatv |
| 1021 | pvivafimlr ayflqtsqql kqlesegrsp ifthlvtslk glwtlrafgr qpyfetlfhk |
| 1081 | alnlhtanwf lylstlrwfq mriemifvif fiavtfisil ttgegegrvg iiltlamnim |
| 1141 | stlqwavnss idvdslmrsv srvfkfidmp tegkptkstk pykngqlskv miienshvkk |
| 1201 | ddiwpsggqm tvkdltakyt eggnaileni sfsispgqrv gllgrtgsgk stllsaflrl |
| 1261 | lntegeiqid gvswdsitlq qwrkafgvip qkvfifsgtf rknldpyeqw sdqeiwkvad |
| 1321 | evglrsvieq fpgkldfvlv dggcvlshgh kqlmclarsv lskakillld epsahldpvt |
| 1381 | yqiirrtlkq afadctvilc ehrieamlec qqflvieenk vrqydsiqkl lnerslfrqa |
| 1441 | ispsdrvklf phrnsskcks kpqiaalkee teeevqdtrl |

By “CFTR polynucleotide” is meant a nucleic acid molecule encoding a CFTR polypeptide, or a fragment thereof. An exemplary CFTR polynucleotide sequence is provided at NCBI reference sequence NO.: NM_000492.4, which is reproduced below:

| (SEQ ID NO: 2) | |
|---|---|
| 1 | gtagtaggtc tttggcatta ggagcttgag cccagacggc cctagcaggg accccagcgc |
| 61 | ccgagagacc atgcagaggt cgcctctgga aaaggccagc gttgtctcca aacttttttt |
| 121 | cagctggacc agaccaattt tgaggaaagg atacagacag cgcctggaat tgtcagacat |
| 181 | ataccaaatc ccttctgttg attctgctga caatctatct gaaaaattgg aaagagaatg |
| 241 | ggatagagag ctggcttcaa agaaaaatcc taaactcatt aatgcccttc ggcgatgttt |
| 301 | tttctggaga tttatgttct atggaatctt tttatattta ggggaagtca ccaaagcagt |
| 361 | acagcctctc ttactgggaa gaatcatagc ttcctatgac ccggataaca aggaggaacg |
| 421 | ctctatcgcg atttatctag gcataggctt atgccttctc tttattgtga ggacactgct |
| 481 | cctacaccca gccatttttg gccttcatca cattggaatg cagatgagaa tagctatgtt |
| 541 | tagtttgatt tataagaaga ctttaaagct gtcaagccgt gttctagata aaataagtat |
| 601 | tggacaactt gttagtctcc tttccaacaa cctgaacaaa tttgatgaag gacttgcatt |
| 661 | ggcacatttc gtgtggatcg ctcctttgca agtggcactc ctcatggggc taatctggga |
| 721 | gttgttacag gcgtctgcct tctgtggact tggtttcctg atagtccttg ccctttttca |
| 781 | ggctgggcta gggagaatga tgatgaagta cagagatcag agagctggga agatcagtga |
| 841 | aagacttgtg attacctcag aaatgattga aaatatccaa tctgttaagg catactgctg |
| 901 | ggaagaagca atggaaaaaa tgattgaaaa cttaagacaa acagaactga aactgactcg |
| 961 | gaaggcagcc tatgtgagat acttcaatag ctcagccttc ttcttctcag ggttctttgt |
| 1021 | ggtgttttta tctgtgcttc cctatgcact aatcaaagga atcatcctcc ggaaaatatt |
| 1081 | caccaccatc tcattctgca ttgttctgcg catggcggtc actcggcaat ttccctgggc |
| 1141 | tgtacaaaca tggtatgact ctcttggagc aataaacaaa atacaggatt tcttacaaaa |
| 1201 | gcaagaatat aagacattgg aatataactt aacgactaca gaagtagtga tggagaatgt |
| 1261 | aacagccttc tgggaggagg gatttgggga attatttgag aaagcaaaac aaaacaataa |
| 1321 | caatagaaaa acttctaatg gtgatgacag cctcttcttc agtaatttct cacttcttgg |
| 1381 | tactcctgtc ctgaaagata ttaatttcaa gatagaaaga ggacagttgt tggcggttgc |
| 1441 | tggatccact ggagcaggca agacttcact tctaatggtg attatgggag aactggagcc |
| 1501 | ttcagagggt aaaattaagc acagtggaag aatttcattc tgttctcagt tttcctggat |
| 1561 | tatgcctggc accattaaag aaaatatcat ctttggtgtt tcctatgatg aatatagata |
| 1621 | cagaagcgtc atcaaagcat gccaactaga agaggacatc tccaagtttg cagagaaaga |
| 1681 | caatatagtt cttggagaag gtggaatcac actgagtgga ggtcaacgag caagaatttc |
| 1741 | tttagcaaga gcagtataca aagatgctga tttgtattta ttagactctc cttttggata |
| 1801 | cctagatgtt ttaacagaaa aagaaatatt tgaaagctgt gtctgtaaac tgatggctaa |
| 1861 | caaaactagg attttggtca cttctaaaat ggaacattta aagaaagctg acaaaatatt |
| 1921 | aattttgcat gaaggtagca gctattttta tgggacattt tcagaactcc aaaatctaca |
| 1981 | gccagacttt agctcaaaac tcatgggatg tgattctttc gaccaattta gtgcagaaag |
| 2041 | aagaaattca atcctaactg agaccttaca ccgtttctca ttagaaggag atgctcctgt |
| 2101 | ctcctggaca gaaacaaaaa aacaatcttt taaacagact ggagagtttg gggaaaaaag |
| 2161 | gaagaattct attctcaatc caatcaactc tatacgaaaa ttttccattg tgcaaaagac |
| 2221 | tcccttacaa atgaatggca tcgaagagga ttctgatgag cctttagaga gaaggctgtc |
| 2281 | cttagtacca gattctgagc agggagaggc gatactgcct cgcatcagcg tgatcagcac |
| 2341 | tggccccacg cttcaggcac gaaggaggca gtctgtcctg aacctgatga cacactcagt |
| 2401 | taaccaaggt cagaacattc accgaaagac aacagcatcc acacgaaaag tgtcactggc |
| 2461 | ccctcaggca aacttgactg aactggatat atattcaaga aggttatctc aagaaactgg |
| 2521 | cttggaaata agtgaagaaa ttaacgaaga agacttaaag gagtgctttt ttgatgatat |
| 2581 | ggagagcata ccagcagtga ctacatggaa cacatacctt cgatatatta ctgtccacaa |
| 2641 | gagcttaatt tttgtgctaa tttggtgctt agtaattttt ctggcagagg tggctgcttc |
| 2701 | tttggttgtg ctgtggctcc ttggaaacac tcctcttcaa gacaaaggga atagtactca |
| 2761 | tagtagaaat aacagctatg cagtgattat caccagcacc agttcgtatt atgtgtttta |
| 2821 | catttacgtg ggagtagccg acactttgct tgctatggga ttcttcagag gtctaccact |
| 2881 | ggtgcatact ctaatcacag tgtcgaaaat tttacaccac aaaatgttac attctgttct |
| 2941 | tcaagcacct atgtcaaccc tcaacacgtt gaaagcaggt gggattctta atagattctc |
| 3001 | caaagatata gcaattttgg atgaccttct gcctcttacc atatttgact tcatccagtt |
| 3061 | gttattaatt gtgattggag ctatagcagt tgtcgcagtt ttacaaccct acatctttgt |
| 3121 | tgcaacagtg ccagtgatag tggcttttat tatgttgaga gcatatttcc tccaaacctc |
| 3181 | acagcaactc aaacaactgg aatctgaagg caggagtcca attttcactc atcttgttac |
| 3241 | aagcttaaaa ggactatgga cacttcgtgc cttcggacgg cagccttact ttgaaactct |
| 3301 | gttccacaaa gctctgaatt tacatactgc caactggttc ttgtacctgt caacactgcg |
| 3361 | ctggttccaa atgagaatag aaatgatttt tgtcatcttc ttcattgctg ttaccttcat |
| 3421 | ttccatttta acaacaggag aaggagaagg aagagttggt attatcctga ctttagccat |
| 3481 | gaatatcatg agtacattgc agtgggctgt aaactccagc atagatgtgg atagcttgat |
| 3541 | gcgatctgtg agccgagtct ttaagttcat tgacatgcca acagaaggta aacctaccaa |
| 3601 | gtcaaccaaa ccatacaaga atggccaact ctcgaaagtt atgattattg agaattcaca |
| 3661 | cgtgaagaaa gatgacatct ggccctcagg gggccaaatg actgtcaaag atctcacagc |
| 3721 | aaaatacaca gaaggtggaa atgccatatt agagaacatt tccttctcaa taagtcctgg |
| 3781 | ccagagggtg ggcctcttgg gaagaactgg atcagggaag agtactttgt tatcagcttt |
| 3841 | tttgagacta ctgaacactg aaggagaaat ccagatcgat ggtgtgtctt gggattcaat |
| 3901 | aactttgcaa cagtggagga aagcctttgg agtgatacca cagaaagtat ttattttttc |
| 3961 | tggaacattt agaaaaaact tggatcccta tgaacagtgg agtgatcaag aaatatggaa |
| 4021 | agttgcagat gaggttgggc tcagatctgt gatagaacag tttcctggga agcttgactt |
| 4081 | tgtccttgtg gatgggggct gtgtcctaag ccatggccac aagcagttga tgtgcttggc |
| 4141 | tagatctgtt ctcagtaagg cgaagatctt gctgcttgat gaacccagtg ctcatttgga |
| 4201 | tccagtaaca taccaaataa ttagaagaac tctaaaacaa gcatttgctg attgcacagt |
| 4261 | aattctctgt gaacacagga tagaagcaat gctggaatgc caacaatttt tggtcataga |
| 4321 | agagaacaaa gtgcggcagt acgattccat ccagaaactg ctgaacgaga ggagcctctt |
| 4381 | ccggcaagcc atcagcccct ccgacagggt gaagctcttt ccccaccgga actcaagcaa |
| 4441 | gtgcaagtct aagccccaga ttgctgctct gaaagaggag acagaagaag aggtgcaaga |
| 4501 | tacaaggctt tagagagcag cataaatgtt gacatgggac atttgctcat ggaattggag |
| 4561 | ctcgtgggac agtcacctca tggaattgga gctcgtggaa cagttacctc tgcctcagaa |
| 4621 | aacaaggatg aattaagttt ttttttaaaa aagaaacatt tggtaagggg aattgaggac |
| 4681 | actgatatgg gtcttgataa atggcttcct ggcaatagtc aaattgtgtg aaaggtactt |
| 4741 | caaatccttg aagatttacc acttgtgttt tgcaagccag attttcctga aaacccttgc |
| 4801 | catgtgctag taattggaaa ggcagctcta aatgtcaatc agcctagttg atcagcttat |
| 4861 | tgtctagtga aactcgttaa tttgtagtgt tggagaagaa ctgaaatcat acttcttagg |
| 4921 | gttatgatta agtaatgata actggaaact tcagcggttt atataagctt gtattccttt |
| 4981 | ttctctcctc tccccatgat gtttagaaac acaactatat tgtttgctaa gcattccaac |
| 5041 | tatctcattt ccaagcaagt attagaatac cacaggaacc acaagactgc acatcaaaat |
| 5101 | atgccccatt caacatctag tgagcagtca ggaaagagaa cttccagatc ctggaaatca |
| 5161 | gggttagtat tgtccaggtc taccaaaaat ctcaatattt cagataatca caatacatcc |
| 5221 | cttacctggg aaagggctgt tataatcttt cacaggggac aggatggttc ccttgatgaa |
| 5281 | gaagttgata tgccttttcc caactccaga aagtgacaag ctcacagacc tttgaactag |
| 5341 | agtttagctg gaaaagtatg ttagtgcaaa ttgtcacagg acagcccttc tttccacaga |
| 5401 | agctccaggt agagggtgtg taagtagata ggccatgggc actgtgggta gacacacatg |
| 5461 | aagtccaagc atttagatgt ataggttgat ggtggtatgt tttcaggcta gatgtatgta |
| 5521 | cttcatgctg tctacactaa gagagaatga gagacacact gaagaagcac caatcatgaa |
| 5581 | ttagttttat atgcttctgt tttataattt tgtgaagcaa aattttttct ctaggaaata |
| 5641 | tttattttaa taatgtttca aacatatata acaatgctgt attttaaaag aatgattatg |
| 5701 | aattacattt gtataaaata atttttatat ttgaaatatt gactttttat ggcactagta |
| 5761 | tttctatgaa atattatgtt aaaactggga caggggagaa cctagggtga tattaaccag |
| 5821 | gggccatgaa tcaccttttg gtctggaggg aagccttggg gctgatgcag ttgttgccca |
| 5881 | cagctgtatg attcccagcc agcacagcct cttagatgca gttctgaaga agatggtacc |
| 5941 | accagtctga ctgtttccat caagggtaca ctgccttctc aactccaaac tgactcttaa |
| 6001 | gaagactgca ttatatttat tactgtaaga aaatatcact tgtcaataaa atccatacat |
| 6061 | ttgtgtgaaa |

In embodiments, the CFTR polynucleotide is a DNA molecule or mRNA molecule.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Non-limiting examples of diseases that may be treated using the compositions and/or methods provided herein include cystic fibrosis, necrotizing enterocolitis, methylmalonic aciduria, alloimmune disorders, metabolic urea disorders, and a Surfactant Protein B (SPB) deficiency, which can include pulmonary alveolar proteinosis.

By “effective amount” is meant the amount of an agent required to ameliorate the symptoms of a disease relative to an untreated patient. In some embodiments, the agent is an mRNA encoding CFTR or SPB. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By “lipopolyplex” is meant a structure for encapsulation and delivery of an agent into an organism and comprises a nucleic acid molecule, cationic lipids, and cationic polymers. Non-limiting examples of lipopolyplex compositions include those described in To et al., Expert Opinion on Drug Discovery 16, 1307-1313, 2021, the disclosure of which is incorporated herein in its entirety for all purposes.

By “lipid nanoparticle” is meant a small particle comprising lipids that is suitable for delivering an agent to a cell. In embodiments, the cell is in a subject. In embodiments, the lipid nanoparticle comprises (e.g., encapsulates) the agent. In various embodiments, the agent is an mRNA molecule. As used herein, the term lipid encompasses triglycerides (e.g. tristearin), diglycerides (e.g. glycerol bahenate), monoglycerides (e.g. glycerol monostearate), fatty acids (e.g. stearic acid), steroids (e.g. cholesterol), and waxes (e.g. cetyl palmitate).

As used herein, “obtaining” as in “obtaining an agent” includes synthesizing, purchasing, or otherwise acquiring the agent.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “surfactant protein B” (SPB) polypeptide is meant a polypeptide or fragment thereof having at least 85% amino acid sequence identity to NCBI reference sequence NO.: NP_000533.4, provided below, or a fragment thereof capable of increasing spreading and/or stability of a surfactant monolayer in a lung.

NP 000533.4

| (SEQ ID NO: 3) | |
|---|---|
| 1 | maeshllqwl llllptlcgp gtaawttssl acaqgpefwc qsleqalqer alghclqevw |
| 61 | ghvgaddlcq ecedivhiln kmakeaifqd tmrkfleqec nvlplkllmp qcnqvlddyf |
| 121 | plvidyfqnq tdsngicmhl glcksrqpep egepgmsdpl pkplrdplpd plldklvlpv |
| 181 | lpgalqarpg phtqdlseqq fpiplpycwl cralikriga mipkgalava vaqvcrvvpl |
| 241 | vaggicqcla erysvilldt llgrmlpqlv crlvlrcsmd dsagprsptg ewlprdsech |
| 301 | lcmsvttqag nsseqaipqa mlqacvgswl drekckqfve qhtpqlltlv prgwdahttc |
| 361 | qalgvcgtms splqcihspd l |

By “SPB polynucleotide” is meant a nucleic acid molecule encoding a SPB polypeptide, or a fragment thereof. An exemplary SPB polynucleotide sequence is provided at NCBI reference sequence NO.: NM_000542.5, which is reproduced below:

| (SEQ ID NO: 4) | |
|---|---|
| 1 | aggctgcaga ggtgccatgg ctgagtcaca cctgctgcag tggctgctgc tgctgctgcc |
| 61 | cacgctctgt ggcccaggca ctgctgcctg gaccacctca tccttggcct gtgcccaggg |
| 121 | ccctgagttc tggtgccaaa gcctggagca agcattgcag tgcagagccc tagggcattg |
| 181 | cctacaggaa gtctggggac atgtgggagc cgatgaccta tgccaagagt gtgaggacat |
| 241 | cgtccacatc cttaacaaga tggccaagga ggccattttc caggacacga tgaggaagtt |
| 301 | cctggagcag gagtgcaacg tcctcccctt gaagctgctc atgccccagt gcaaccaagt |
| 361 | gcttgacgac tacttccccc tggtcatcga ctacttccag aaccagactg actcaaacgg |
| 421 | catctgtatg cacctgggcc tgtgcaaatc ccggcagcca gagccagagc aggagccagg |
| 481 | gatgtcagac cccctgccca aacctctgcg ggaccctctg ccagaccctc tgctggacaa |
| 541 | gctcgtcctc cctgtgctgc ccggggccct ccaggcgagg cctgggcctc acacacagga |
| 601 | tctctccgag cagcaattcc ccattcctct cccctattgc tggctctgca gggctctgat |
| 661 | caagcggatc caagccatga ttcccaaggg tgcgctagct gtggcagtgg cccaggtgtg |
| 721 | ccgcgtggta cctctggtgg cgggcggcat ctgccagtgc ctggctgagc gctactccgt |
| 781 | catcctgctc gacacgctgc tgggccgcat gctgccccag ctggtctgcc gcctcgtcct |
| 841 | ccggtgctcc atggatgaca gcgctggccc aaggtcgccg acaggagaat ggctgccgcg |
| 901 | agactctgag tgccacctct gcatgtccgt gaccacccag gccgggaaca gcagcgagca |
| 961 | ggccatacca caggcaatgc tccaggcctg tgttggctcc tggctggaca gggaaaagtg |
| 1021 | caagcaattt gtggagcagc acacgcccca gctgctgacc ctggtgccca ggggctggga |
| 1081 | tgcccacacc acctgccagg ccctcggggt gtgtgggacc atgtccagcc ctctccagtg |
| 1141 | tatccacagc cccgaccttt gatgagaact cagctgtcca gctgcaaagg aaaagccaag |
| 1201 | tgagacgggc tctgggacca tggtgaccag gctcttcccc tgctccctgg ccctcgccag |
| 1261 | ctgccaggct gaaaagaagc ctcagctccc acaccgccct cctcaccgcc cttcctcggc |
| 1321 | agtcacttcc actggtggac cacgggcccc cagccctgtg tcggccttgt ctgtctcagc |
| 1381 | tcaaccacag tctgacacca gagcccactt ccatcctctc tggtgtgagg cacagcgagg |
| 1441 | gcagcatctg gaggagctct gcagcctcca cacctaccac gacctcccag ggctgggctc |
| 1501 | aggaaaaacc agccactgct ttacaggaca gggggttgaa gctgagcccc gcctcacacc |
| 1561 | cacccccatg cactcaaaga ttggatttta cagctacttg caattcaaaa ttcagaagaa |
| 1621 | taaaaaatgg gaacatacag aactctaaaa gatagacatc agaaattgtt aagttaagct |
| 1681 | ttttcaaaaa atcagcaatt ccccagcgta gtcaagggtg gacactgcac gctctggcat |
| 1741 | gatgggatgg cgaccgggca agctttcttc ctcgagatgc tctgctgctt gagagctatt |
| 1801 | gctttgttaa gatataaaaa ggggtttctt tttgtctttc tgtaaggtgg acttccagct |
| 1861 | tttgattgaa agtcctaggg tgattctatt tctgctgtga tttatctgct gaaagctcag |
| 1921 | ctggggttgt gcaagctagg gacccattcc tgtgtaatac aatgtctgca ccaatgctaa |
| 1981 | taaagtccta ttctctttta tgagaaagaa aaagacaccg tcctttaaag tgctgcagta |
| 2041 | tggccagacg tggtggctca cacctgcaat cccagcacct taggaggccg aggcaggagg |
| 2101 | atccttgagg tcaggagttc gagaccagcc tcgccaacat ggtgaaaccc catttctact |
| 2161 | aaaaatacaa aaaattagcc aagtgtggtg gcatatgcct gtaatcccaa ctactcagaa |
| 2221 | ggccgaggca ggagaattac ttgaacgcag gagaatcact gcagcccagg aggcagaggt |
| 2281 | tgcagtgagc cgagattgca ccactgcact ccagcctggg tgacagagca agactccatc |
| 2341 | tcagtaaata aataaataaa taaaaagcgc tgcagtagct gtggcctcac cctgaagtca |
| 2401 | gcgggcccag gcctacctca ctctctccct tggcagagaa gcagacgtcc atagctcctc |
| 2461 | tccctcacaa gcgctcccag cctgccctcc agctgctgct ctcccctccc agtctctact |
| 2521 | cactgggatg aggttaggtc atgaggacac caaaaaccta aaaataaaca aaaagccaaa |
| 2581 | caagccttag cttttcttaa agactgaaat gcctggaagt gtccctttat ttataaaata |
| 2641 | acttttgtca tatttcttat acatgtttct tgtaagaaat tcagaaacta cagacaaaga |
| 2701 | gagtggaaat tacccactgt caggcctctg agcccaagct aagccatcat atcccctgtg |
| 2761 | ccctgcacgt atacacccag atggcctgaa gcaactgaag atccacaaaa gaagtgaaaa |
| 2821 | tagccagttc ctgccttaac tgatgacatt ccaccattgt gatttgttcc tgccccaccc |
| 2881 | taactgatca attgaccttg tgacaataca ccttccccac ccttgagaag gtgctttgta |
| 2941 | atattctccc cacccacccc acgcccgcac ccccgcaccc ttaagaaggt attttgtaat |
| 3001 | attctctccg ccattgagaa tgtgctttgt aagatccacc ccctgcccac aaaaaattgc |
| 3061 | tcctaactcc accgcctatc ccaaacctac aagaactaat gataatccca ccaccctttg |
| 3121 | ctgactcttt ttggactcag cccacctgca cccaggtgat taaaaagctt tattgttcac |
| 3181 | acaaagcctg tttggtagtc tcttcacagg gaagcatgtg acacccacaa tcccacctag |
| 3241 | cccaggagag agctacggca gggtgtgtgt tttgacactg agcttggggc tttttccatc |
| 3301 | ttctccccac agcctctggc tccacacctc caccgttcaa gcgccagaaa gagctgtcta |
| 3361 | tgcagcctgc tcttgggcct ggggatgaga cacacaattc attggctcct ggattttaag |
| 3421 | tagacatttg taaatctata gctaactact gtccttaaag ccattgtttc cattacaaaa |
| 3481 | tccaactctc tgagagaaaa gggtgtttta aatttaaaaa aataaaaaca aaaaagtttg |
| 3541 | attgagacaa |

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule (e.g., mRNA or an RNA molecule) that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. In some instances, the nucleic acid molecule is an mRNA molecule. In embodiments, the mRNA molecule comprises a 5′ cap, a poly (A) tail, a 3′ untranslated region, and/or a 5′ untranslated region. Components of mRNA molecules suitable for use in the methods of the present invention include those described in Sahin, et al., “mRNA-based therapeutics-developing a new class of drugs,” Nature Reviews Drug Discovery, 13:759-780 (2014), the disclosure of which is incorporated herein by reference in its entirety for all purposes.

In embodiments, the polynucleotides provided herein contain one or more modifications or analogs. For example, in some embodiments a polynucleotide contains one or more analogs (e.g., altered backbone, sugar, or nucleobase). Some non-limiting examples of analogs include 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. In embodiments, the polynucleotide contains a modified backbone and/or linkages (e.g., between adjacent nucleosides). Non-limiting examples of modified backbones include those that contain a phosphorus atom in the backbone and those that do not contain a phosphorus atom in the backbone. Non-limiting examples of modified backbones include phosphorothioates, chiral phosphorothioates, phosphorodithioates, phosphotriesters, aminoalkyl phosphotriesters, methyl and other alkyl phosphonate such as 3′-alkylene phosphonates, 5′-alkylene phosphonates, chiral phosphonates, phosphinates, phosphoramidates including 3′-amino phosphoramidate and aminoalkyl phosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage. In embodiments, a polynucleotide contains short chain alkyl or cycloalkyl linkages (e.g., between adjacent nucleosides), mixed heteroatom and alkyl or cycloalkyl internucleoside linkages, or one or more short chain heteroatomic or heterocyclic internucleoside linkages. In embodiments, a polynucleotide includes one or more of the following: morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. In embodiments, a polynucleotide contains a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. In embodiments, the backbone in PNA compounds contains two or more linked aminoethylglycine units that give PNA an amide containing backbone. Heterocyclic base moieties can be bound directly or indirectly to aza nitrogen atoms of the amide portion of the backbone.

In embodiments, a polynucleotide contains a morpholino backbone structure. For example, a nucleic acid can contain a 6-membered morpholino ring in place of a ribose ring. In some of these embodiments, a phosphorodiamidate or other non-phosphodiester internucleoside linkage can replace a phosphodiester linkage.

A polynucleotide can contain linked morpholino units having heterocyclic bases attached to the morpholino ring. Linking groups can link morpholino monomeric units. Non-ionic morpholino-based oligomeric compounds can have less undesired interactions with cellular proteins. Morpholino-based polynucleotides can be nonionic mimics of nucleic acids. A variety of compounds within the morpholino class can be joined using different linking groups. A further class of polynucleotide mimetic can be referred to as cyclohexenyl nucleic acids (CeNA). In some instances, the furanose ring normally present in a nucleic acid molecule is replaced with a cyclohexenyl ring. CeNA DMT protected phosphoramidite monomers can be prepared and used for oligomeric compound synthesis using phosphoramidite chemistry. In some cases, incorporation of CeNA monomers into a nucleic acid chain increases the stability of a DNA/RNA hybrid. CeNA oligoadenylates can form complexes with nucleic acid complements with similar stability to the native complexes. In embodiments, a polynucleotide contains Locked Nucleic Acids (LNAs) in which the 2′-hydroxyl group is linked to the 4′ carbon atom of the sugar ring thereby forming a 2′-C, 4′-C-oxymethylene linkage, thereby forming a bicyclic sugar moiety. The linkage can be a methylene (—CH₂), group bridging the 2′ oxygen atom and the 4′ carbon atom wherein n is 1 or 2. LNA and LNA analogs can display very high duplex thermal stabilities with complementary nucleic acid (Tₘ=+3 to +10° C.), stability towards 3′-exonucleolytic degradation and good solubility properties.

In embodiments, a polynucleotide contains nucleobase modifications (often referred to simply as “base modifications”) or substitutions. In embodiments, unmodified nucleobases include one or more of the purine bases, (e.g., adenine (A) and guanine (G)), and/or the pyrimidine bases, (e.g., thymine (T), cytosine (C) and uracil (U)). Non-limiting examples of modified nucleobases include nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Further non-limiting examples of modified nucleobases include tricyclic pyrimidines such as phenoxazine cytidine (1H-pyrimido (5,4-b) (1,4) benzoxazin-2 (3H)-one), phenothiazine cytidine (1H-pyrimido (5,4-b) (1,4) benzothiazin-2 (3H)-one), G-clamps such as a substituted phenoxazine cytidine (e.g., 9-(2-aminoethoxy)-H-pyrimido (5,4-(b) (1,4) benzoxazin-2 (3H)-one), phenothiazine cytidine (1H-pyrimido (5,4-b) (1,4) benzothiazin-2 (3H)-one), G-clamps such as a substituted phenoxazine cytidine (e.g., 9-(2-aminoethoxy)-H-pyrimido (5,4-(b) (1,4) benzoxazin-2 (3H)-one), carbazole cytidine (2H-pyrimido (4,-b) indol-2-one), pyridoindole cytidine (H-pyrido (3′,2′: 4, 5) pyrrolo[2,3-d]pyrimidin-2-one).

By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York. By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 provides a schematic diagram showing an overview of gene insertion and luminometry in fetal sites and maternal serum. E=gestational day.

FIGS. 2A-2D provide plots showing relative luminescence for several insertion sites compared to controls at E18 and E21. FIG. 2A provides a plot showing a comparison of relative luminescence in control and mRNA in umbilical cord or chorion or placenta. FIG. 2B provides a plot showing a comparison of relative luminescence in control and mRNA in amnion. FIG. 2C provides a plot showing a comparison of relative luminescence in control and mRNA in amniotic fluid or fetal serum. FIG. 2D provides a plot showing luminescence levels in fetal stomach, intestines, lung and liver. E=gestational day; RLU=relative light unit; values expressed as mean±SEM; * p<0.05 vs. E18 control; ** p<0.05 vs. E21 control.

FIG. 3A provides a representative overview of the experimental protocol. hSPB=human Surfactant protein B; E=gestational day (term=E21). FIG. 3B provides bar graphs showing the survival of fetal rats at E18-E21 for mRNA and control groups. Data presented as absolute numbers, * p<0.05 vs. control, E=gestational day. FIGS. 3C and 3D provide plots comparing relative concentrations of bioactive components in mRNA and control groups. FIG. 3C provides plots showing human surfactant protein B levels in rat fetal lungs at E18-E21 for the mRNA and control groups. FIG. 3D provides plots showing phosphatidylcholine levels in rat amniotic fluid at E18-E21 for the mRNA and control groups. Data was presented as median (interquartile range), * p<0.05 vs control, E=gestational day.

FIG. 4 provides a schematic diagram showing an overview of the experimental protocol for a 21-day gestational term, where “E” represents the gestational day.

FIGS. 5A-5B provide plots showing human cystic fibrosis transmembrane conductance regulator (hCFTR) protein levels by enzyme-linked immunosorbent assay (ELISA) in fetal rats at gestational days 18-21 for the mRNA group, and day 21 only for the control group in small intestine for FIG. 5A and lungs for FIG. 5B. Data is presented as mean±SEM, * p<0.05 vs. Control.

FIG. 6 provides representative images of fluorescence microscopy of fetal small intestine from the mRNA group at gestation day 20 and the control group at gestational day 21, where tissue sections were stained for hCFTR (anti-hCFTR, cy3; light gray) and nuclei (DAPI, dark gray). Magnification: 63X. Scale bar: 40 μm.

### DETAILED DESCRIPTION OF THE INVENTION

The invention features compositions and methods that are useful for transamniotic delivery of mRNA encoding the cystic fibrosis transmembrane conductance regulator protein or surfactant protein B to a fetus for the prenatal treatment of cystic fibrosis or congenital surfactant protein B deficiencies, respectively.

The invention of the disclosure is based, at least in part, upon the discovery that packaged exogenous mRNA can be incorporated by the fetus after simple intra-amniotic administration in a rodent model. As described further in the Examples provided herein, experiments were undertaken to examine the pharmacokinetics of exogenous encapsulated mRNA delivered transamniotically, utilizing a healthy rat model. The pattern and chronology of mRNA incorporation were compatible with transplacental hematogenous routing, as well as with fetal swallowing/aspiration. Controlled by the encapsulating composite without mRNA, luciferase activity was detected in animals that received encapsulated luciferase mRNA in the following fetal annexes: amniotic fluid, amnion, chorion, umbilical cord, and placenta (p=0.033 to <0.001), as well as in the following fetal sites: liver, stomach, intestines, and lungs (p=0.043 to 0.002).

The invention of the disclosure is also based, at least in part, upon the discovery that encapsulated exogenous mRNA encoding for surfactant protein B was incorporated and translated by fetal lung cells following simple intra-amniotic injection in a healthy rat model. The invention of the disclosure is also based, at least in part, upon the discovery that encapsulated exogenous mRNA encoding for the cystic fibrosis transmembrane conductance regulator protein was incorporated and translated by fetal small bowel cells after simple intra-amniotic injection in a healthy rat model.

Many prenatally diagnosable diseases involve abnormal or missing proteins and thus could be conceivably amenable to mRNA-based therapies, along with certain gestational or perinatal infections for which mRNA-based vaccines could also be beneficial (Alapati et al., Science Translational Medicine 11, 2019; Antony et al., Molecular and Cellular Pediatrics 2, 11, 2015; Derosa et al., Gene Therapy 23, 699-707, 2016; Fazio et al., Gene therapy 11, 544-551, Kang et al., Nature Communications 11, 3929, 2020; Massaro et al., Nature Medicine 24, 1317-1323, 2018; Riley et al., Science Advances 7, 2021; Rizzi et al, Vaccine 23, 4273-4282, 2005, the disclosures of which are incorporated herein by reference in their entireties for all purposes).

### Cystic Fibrosis

Mutations of the cystic fibrosis transmembrane conductance regulator (CFTR), a cell membrane protein serving as chloride/anion channel in numerous epithelial cells, constitute the central component of the pathophysiology of cystic fibrosis (CF) and chief cause of neonatal meconium ileus associated with this disease. Postnatal exogenous mRNA-based therapies for CF have been widely studied experimentally, having already reached clinical trials as inhalation therapies for its respiratory complications. It has been previously shown that the transamniotic route can be an effective means of delivering encapsulated exogenous mRNA to the fetus, as a variant of the transamniotic nucleic acid therapy (TRANAT) principle. Researchers sought to determine whether TRANAT could be a viable alternative for the administration of CFTR mRNA to the fetal bowel as a potential strategy for the perinatal management of meconium ileus associated with CF.

As a monogenic autosomal recessive disease, cystic fibrosis (CF) is a candidate for novel therapies based on exogenous mRNA administration. A central component of CF pathophysiology is a mutation in the CF transmembrane conductance regulator (CFTR), a cell membrane protein serving as chloride/anion channel in numerous cell phenotypes, leading to CFTR absence or malfunction. The gastrointestinal tract is commonly affected and as early as in the perinatal period, so that newborns with CF may suffer from meconium ileus as a result of that, followed by respiratory complications not long thereafter. The diagnosis of CF is often made early in pregnancy via genetic screening. In addition, certain fetal ultrasound findings may be predictive of meconium ileus after birth (Scotet et al., Am J Obstet Gynecol 203, 592 e1-6, 2010, the disclosure of which is incorporated herein by reference in its entirety for all purposes).

### Congenital Surfactant Protein B Deficiency

Surfactant protein B (SPB) plays a central role in surfactant production, stability and function. Congenital diseases, such a SPB deficiency and pulmonary alveolar proteinosis (PAP), involve the absence of SPB and are associated with very high mortality. In preterm infants, surfactant deficiency leads to respiratory distress and significant morbidity/mortality. SFB deficiencies are inherited in an autosomal recessive manner through mutations in the SFTPB gene, which can be detected through genetic testing of the parents. SPB deficiency is typically diagnosed using genetic testing to identify SPB deficiency-associated mutations in a subject.

### Transamniotic Nucleic Acid Therapy

The invention provides transamniotic nucleic acid therapy (TRANAT) methods for treating cystic fibrosis and/or treating and/or preventing a surfactant protein B deficiency preventing necrotizing enterocolitis or symptoms thereof. Thus, one embodiment is a method of treating a fetus suffering from or susceptible to a disease (e.g., cystic fibrosis), infection, and/or disorder or symptom thereof. The methods involve administering to the amniotic fluid a therapeutically effective amount of an mRNA. The mRNA is administered by intra-amniotic injection. The method involves the step of administering to the fetus a therapeutic amount of an amount of an mRNA sufficient to treat the disease or disorder or symptom thereof, under conditions such that the disease, infection, and/or disorder is treated and/or prevented. The mRNA can be delivered using common microencapsulation methods utilizing lipid nanoparticle delivery such as, for example, those described in: “Hou et al., “Lipid Nanoparticles for MRNA Delivery,” Nature Reviews Materials, December 2021, Vol. 6, No. 12, pp. 1078-1094.” In some embodiments, the method of delivery can include the use of lipopolyplexes such as, for example, those described in: “Bofinger et al., “Development of Lipopolyplexes for Gene Delivery: A Comparison of the Effects of Differing Modes of Targeting Peptide Display on the Structure and Transfection Activities of Lipopolyplexes,” Journal of Peptide Science, 2018, Vol. 24, No. 12, p. e3131.”

The methods herein include administering to the amniotic fluid surrounding a fetus an effective amount of an mRNA, or a composition described herein to produce such effect. Identifying a subject in need of such treatment can be in the judgment of a subject or a health care professional and can be subjective (e.g., opinion) or objective (e.g., measurable by a test or diagnostic method).

The therapeutic methods of the invention (which include prophylactic treatment) in general comprise administration of a therapeutically effective amount of an mRNA to the amniotic fluid surrounding a fetus. In embodiments, the fetus is a human fetus. Such treatment will be suitably administered to fetuses, suffering from, having, susceptible to, or at risk for a disease, infection, and/or disorder, or symptom thereof, before and/or after birth. Determination of those subjects “at risk” can be made by any objective or subjective determination by a diagnostic test or opinion of a subject or health care provider (e.g., genetic test, enzyme or protein marker, Marker (as defined herein), family history, and the like).

In some embodiments, an mRNA is administered to the amniotic fluid in an amount of about or at least about 0.01 μg, 0.1 μg, 0.5 μg, 1 μg, 5 μg, 10 μg, 15 μg, 20 μg, 25 μg, 100 μg, 250 μg, 500 μg, 750 μg, 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 100 mg, 200 mg, 300 mg, 400 mg, 500 mg, 600 mg, 700 mg, 800 mg, 900 mg, or 1,000 mg. In some embodiments, an mRNA is administered to the amniotic fluid in an amount of no more than about 0.01 μg, 0.1 μg, 0.5 μg, 1 μg, 5 μg, 10 μg, 15 μg, 20 μg, 25 μg, 100 μg, 250 μg, 500 μg, 750 μg, 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 200 mg, 300 mg, 400 mg, 500 mg, 600 mg, 700 mg, 800 mg, 900 mg, or 1,000 mg.

Generally, doses of mRNAs of the invention will be from about 0.0001 mg/kg fetal weight per day to about 1000 mg/kg fetal weight per administration. It is expected that doses ranging from about 1 to about 50 mg/kg will be suitable. In an embodiment, an mRNA is administered to the amniotic fluid in amounts sufficient to deliver about or at least about 0.1 mg/kg fetal weight, 0.0001 mg/kg fetal weight, 0.001 mg/kg fetal weight, 0.01 mg/kg fetal weight, 0.1 mg/kg fetal weight, 0.5 mg/kg fetal weight, 1 mg/kg fetal weight, 2 mg/kg fetal weight, 3 mg/kg fetal weight, 4 mg/kg fetal weight, 5 mg/kg fetal weight, 6 mg/kg fetal weight 7 mg/kg fetal weight, 8 mg/kg fetal weight, 9 mg/kg fetal weight, or 10 mg/kg fetal weight of the polynucleotide to the fetus. In an embodiment, an mRNA is administered to the amniotic fluid in amounts sufficient to deliver no more than about 0.0001 mg/kg fetal weight, 0.001 mg/kg fetal weight, 0.01 mg/kg fetal weight, 0.1 mg/kg fetal weight, 05 mg/kg fetal weight, 1 mg/kg fetal weight, 2 mg/kg fetal weight, 3 mg/kg fetal weight, 4 mg/kg fetal weight, 5 mg/kg fetal weight, 6 mg/kg fetal weight 7 mg/kg fetal weight, 8 mg/kg fetal weight, 9 mg/kg fetal weight, or 10 mg/kg fetal weight of the polynucleotide to the fetus. In the event that a response in a subject is insufficient at the initial doses applied, higher doses (or effectively higher doses) may be employed to the extent that subject tolerance permits. Multiple doses per day are contemplated to achieve appropriate systemic levels of an agent and/or compositions of the invention.

In some embodiments, administration of an mRNA of the invention to the amniotic fluid is associated with a reduction in the intensity, severity, or frequency, or delays the onset of a disorder (e.g., cystic fibrosis), disease, and/or deficiency (e.g., a surfactant protein B deficiency) in the fetus before and/or after birth (i.e., prenatal or perinatal).

It can be advantageous to administer the mRNA at a particular time during the development of the fetus. In embodiments, the mRNA is administered prior to, at, after, and/or until about 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 25 weeks, 30 weeks, 35 weeks, or 40 weeks of pregnancy. In embodiments, it is advantageous to administer the mRNA prior to 20 weeks of pregnancy.

In embodiments, the trans-amniotic delivery of the mRNA to the amniotic fluid results in the presence of and/or an increase in levels of the mRNA in the fetal blood plasma. In embodiments, the trans-amniotic delivery of the mRNA to the amniotic fluid results in an increase in levels of the mRNA in fetal tissues (e.g., thymus, spleen, brain, and/or bone marrow, and/or others, given the presence of the mRNA in the fetal blood plasma). Further non-limiting examples of tissues include liver, stomach, intestines, pancreas, spleen, thymus, lymph nodes, brain, meninges, heart, blood vessels, lungs, airways, kidneys, ureters, urethra, ovaries, testicles, genitalia, skin, skin annexes, muscle, bone, cartilage, bone marrow, eyes, ears, mouth, nose, pharynx, larynx, vitreous body, cerebrospinal fluid, and peripheral blood.

A skilled practitioner will readily be able to identify suitable methods for detecting and measuring levels of an mRNA in the amniotic fluid or in any samples taken from a fetus or from a neonate that received such treatment before birth. For example, quantitative real-time polymerase chain reaction (qRT-PCR) is one example of many techniques available for measuring mRNA levels in biological samples such as, to provide non-limiting examples, serum, bone marrow, spleen tissue, thymus tissue, and brain tissue. The biological samples can be collected from the fetus and/or the mother.

mRNA

The methods of the invention involve administering an mRNA to the amniotic fluid surrounding a fetus. In embodiments, the mRNA is encapsulated in a lipid nanoparticle or lipopolyplex to enhance stability, tolerability, or solubility; or decrease the likelihood of eliciting an immune response in the target. Additionally, in some embodiments, the invention may include modified nucleobases for masking the nucleic acid payload from the immune system and decreasing the chances of an unintended immune response or triggering anaphylaxis. In some embodiments, such modifications can include, but are not limited to those modifications described herein, such as pseudouridine, thiouridine, and 5-methylcytidine. In some embodiments, the mRNA is single-stranded. In further embodiments, the mRNA can include untranslated regions (UTRs) at the 5′ and 3′ end to enhance stability. In some embodiments, the mRNA includes one or more of a 3′ poly(A) tail, a 5′ cap, UTRs, and a protein-encoding region. In embodiments, the mRNA molecule comprises a 5′ cap, a poly (A) tail, a 3′ untranslated region, and/or a 5′ untranslated region. Components of mRNA molecules suitable for use in the methods of the present invention include those described in Sahin, et al., “mRNA-based therapeutics-developing a new class of drugs,” Nature Reviews Drug Discovery, 13:759-780 (2014), the disclosure of which is incorporated herein by reference in its entirety for all purposes.

In some embodiments, a DNA template can be used to produce synthetic mRNA. In further embodiments, in vitro transcription technology is used to transcribe the sequence of interest into mRNA, which can be purified for delivery into a target or fetus. In some embodiments, mRNA synthesis takes place in a cell-free system. In other embodiments, cells engineered to express an mRNA sequence of interest can be cultivated and purified. In some embodiments, the engineered cells have been transfected with a plasmid containing a DNA template for the mRNA sequence of interest.

In some embodiments, the mRNA being administered to a subject has been modified to improve translation efficiency in a target cell or has been modified to augment expression, which can include optimization of stop and start codons for efficient elongation and termination of translation. This optimization can include the replacement of similar codons with a major codon that is preferred by highly expressed genes or a codon that is decoded by more tRNA than similar codons. Methods for codon optimization are well known and include those methods described, for example, in Mauro and Chappel, “A critical analysis of codon optimization in human therapeutics,” Trends Mol Med., 20:604-613 (2014), the disclosure of which is incorporated herein by reference in its entirety for all purposes.

Pharmaceutical Compositions The invention provides compositions comprising a polynucleotide (e.g., mRNA) for use in methods for preventing and/or treating cystic fibrosis or surfactant protein B deficiencies in a developing fetus. The compositions should be sterile and contain a therapeutically effective amount of the polynucleotide in a unit of weight or volume suitable for administration to a subject.

Agents (e.g., mRNA) of the invention may be administered within a pharmaceutically-acceptable diluent, carrier, or excipient, in unit dosage form. In some embodiments, the agents of the invention may be administered through injection of lipid nanoparticles or lipoplexes containing mRNA of interest.

Suitable pharmaceutically acceptable carriers include but are not limited to water, salt solutions (e.g., NaCl), saline, buffered saline, alcohols, glycerol, ethanol, gum arabic, benzyl alcohols, polyethylene glycols, gelatin, carbohydrates such as lactose, amylose or starch, sugars such as mannitol, sucrose, or others, dextrose, magnesium stearate, viscous paraffin, fatty acid esters, etc., as well as combinations thereof. The pharmaceutical preparations can, if desired, be mixed with auxiliary agents (e.g., lubricants, preservatives, stabilizers, wetting agents, emulsifiers, salts for influencing osmotic pressure, buffers, and/or coloring substances and the like) which do not deleteriously react with the active compounds or interfere with their activity. In an embodiment, a water-soluble carrier suitable for intravenous administration is used.

A suitable pharmaceutical composition, if desired, can also contain minor amounts of wetting or emulsifying agents, or pH buffering agents. A composition can be a liquid solution. Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful delivery systems for agents of the invention include ethylene-vinyl acetate copolymer particles, osmotic pumps, implantable infusion systems, and liposomes.

The formulations can be administered to human patients in therapeutically effective amounts. The preferred dosage of a polynucleotide the invention is likely to depend on such variables as the volume of the amniotic cavity, or the nature of a disorder being treated. In an embodiment, the polynucleotide is administered more than once in a given pregnancy.

A pharmaceutical composition or medicament can be formulated in accordance with the routine procedures as a pharmaceutical composition adapted for administration to human beings. For example, in some embodiments, a composition for intravenous administration typically is a solution in sterile isotonic aqueous buffer. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline, or dextrose/water. Where the composition is administered by injection, an ampule of sterile water for injection or saline can be provided so that the ingredients may be mixed prior to administration.

### Kits

The invention provides kits for use in preventing and/or treating cystic fibrosis and/or to treat and/or prevent surfactant protein B deficiencies in a developing fetus. In one embodiment, the kit contains a pharmaceutical composition containing an mRNA suitable for us in preventing and/or treating cystic fibrosis and/or congenital surfactant protein B deficiency. In an embodiment, the kit contains equipment (e.g., hypodermic needles and syringes) to aid in administration of compositions of the invention to amniotic fluid surrounding a fetus.

Optionally, the kit includes directions for administering the pharmaceutical composition to amniotic fluid. In other embodiments, the kit comprises a sterile container which contains the pharmaceutical composition. Such containers can be boxes, ampules, bottles, vials, tubes, bags, pouches, blister-packs, or other suitable container form known in the art. Such containers can be made of plastic, glass, laminated paper, metal foil, or other materials suitable for holding compositions containing mRNA. The instructions will generally include information about the pharmaceutical composition (e.g., safety information, recommended doses, and the like) and how to administer the composition to the amniotic fluid surrounding a fetus. In other embodiments, the instructions include at least one of the following: description of the mRNA; methods for using the enclosed materials; precautions; warnings; indications; clinical or research studies; and/or references. The instructions may be printed directly on the container (when present), or as a label applied to the container, or as a separate sheet, pamphlet, card, or folder supplied in or with the container.

The practice of the invention employs, unless otherwise indicated, conventional techniques of molecular biology (including recombinant techniques), microbiology, cell biology, biochemistry, and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for specific embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Hematogenous Routing of Exogenous mRNA Delivered into the Amniotic Fluid

Exogenous mRNA administration has recently emerged as a powerful potential or actual therapy for a variety of diseases, based on the replacement of different missing proteins, or on targeted antigen presentation. Many prenatally diagnosable diseases involve abnormal or missing proteins and thus could be conceivably amenable to mRNA-based therapies, along with certain gestational or perinatal infections for which mRNA-based vaccines could also be beneficial. Accordingly, experiments were undertaken to evaluate the pharmacokinetics of exogenous encapsulated mRNA delivered transamniotically, utilizing a healthy rat model to determine whether mRNA could be delivered to a fetus prenatally through transamniotic administration.

mRNA Lipopolyplex Preparation and Characterization

Commercially available firefly luciferase mRNA (APExBIO Technology, Houston, TX) was encapsulated using the TransIT-mRNA transfection kit (Mirus Bio, Madison, WI), which consists of a lipid- and synthetic cationic polymer-based composite (a lipopolyplex) with two components, namely a TransIT Reagent proper and a mRNA Boost Reagent. In accordance with the manufacturer's instructions, 1 μg of mRNA (1 μg/μL stock) was suspended in 45 μL of phosphate-buffered saline (PBS). After that, 2 μL of mRNA Boost Reagent and 2 μL of TransIT Reagent were added to the mixture. The final solution was pipetted gently and incubated for 5-10 minutes at room temperature prior to injection in vivo, so as to allow for mRNA lipopolyplex formation.

Encapsulation efficiency of mRNA into the lipopolyplexes was measured using Quant-iT RiboGreen assay (Thermo Fisher Scientific, Waltham, MA) as previously described (Jiang et al., Biomaterials 176, 122-130, 2018). Briefly, samples of naked mRNA in PBS (1 μg/50 μL stock) were plated along with mRNA encapsulated using TransIT into lipopolyplexes (1 μg/50 μL stock) and mRNA standards in a 96-well plate. Fluorescent RiboGreen reagent (Thermo Fisher Scientific, Waltham, MA) was then added, and the resulting fluorescence intensity was measured on a microplate reader (BMG Labtech, Cary, NC) at 490 nm and 520 nm excitation and emission, respectively, in order to quantify the amount of free mRNA in solution. Encapsulation efficiency was calculated by subtracting the amount of free mRNA from the total originally used to generate the mRNA lipopolyplexes and expressed as a percentage.

### Intra-Amniotic Injections

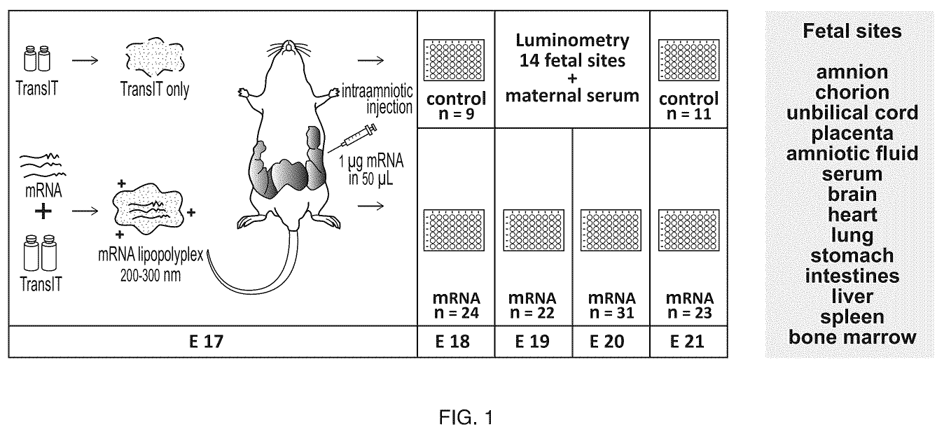

FIG. 1 provides an overview of the experimental design. Ten time-dated pregnant Sprague-Dawley dams (Charles River Laboratories, Wilmington, MA) underwent intra-amniotic injections surgically, under direct vision, on gestational day 17 (E17, term=E21-22) as previously described (Dionigi et al., Journal of Pediatric Surgery 50, 69-73, 2015). In brief, animals were anesthetized and maintained on 2-4% inhaled isoflurane (Patterson Veterinary, Greeley, CO) in 100% oxygen. A midline laparotomy was performed exposing the bicornuate uterus. The amniotic cavities of all viable fetuses (therefore of both sexes) received volume-matched (50 μL) injections of a solution in PBS containing either 1 μg of firefly luciferase mRNA encapsulated in a synthetic lipopolyplex as described above (mRNA group; n=100), or of the lipopolyplex components without any mRNA (control group; n=20) using a 33G non-coring needle on a 100 μL syringe (both from Hamilton Company, Reno, NV). Injections were performed under direct vision with care to avoid injury to the fetus, placenta, or umbilical cord. The incision was closed in two layers. Powdered metronidazole (Unichem Pharmaceuticals, Hasbrouck Heights, NJ) was applied to the wound and sustained release buprenorphine (Zoopharm, Windsor, CO) was administered for post-operative analgesia.

### Specimen Procurement and Processing

Dams were euthanized by CO2 chamber at daily time points from E18 to E21 for the mRNA group and on E18 and E21 for the control group. The laparotomy was reopened, uterus was eviscerated and each fetus was removed en caul. Samples from the following sites were obtained: amniotic fluid, amnion, chorion, umbilical cord, peripheral blood, placenta, liver, stomach, intestines, lung, heart, and brain, as previously described (Lazow et al., Journal of Pediatric Surgery 56, 1233-1236, 2021). Additionally, bone marrow and spleen samples were obtained only from fetuses euthanized at E20 and E21 due to size restraints at earlier gestation ages, as previously described (Lazow et al., Journal of Pediatric Surgery 56, 1233-1236, 2021). Maternal blood was aspirated via direct intracardiac puncture. Fetal and maternal blood samples were centrifuged at 5000 rcf for 8 minutes and serum was obtained from the supernatant. All samples were then rapidly frozen in dry ice-ethanol baths and stored at −80° C. until further processing.

### Luminescence Analysis

Prior to the luminometry assays, all samples except amniotic fluid, bone marrow and serum were weighed and larger ones were normalized not to exceed 200 mg. Lysis buffer (Promega, Madison, WI) was added to each sample in different amounts based on the mass of the sample, as follows: 0.0-9.9 mg received 100 μL; 10.0-39.9 mg received 150 μL; 40.0-69.9 mg received 200 μL; 70.0-99.9 received 250 μL; and 100.0-200.0 mg received 300 μL. Stainless steel lysis beads (Next Advance, Averill Park, NY) were then added and samples underwent homogenization in an automated tissue homogenizer (Next Advance) for 5 minutes, followed by 10 minutes incubation at room temperature. Amniotic fluid and serum samples were vortexed for 5 seconds, incubated for 20 minutes at room temperature with 150 μL of lysis buffer and briefly vortexed again. Bone marrow samples were incubated with 90 μL of lysis buffer. All specimens were then centrifuged at 12,000 rcf for 5 minutes, and the supernatant was isolated for luminescence analysis.

The presence of firefly luciferase, a product of the exogenous mRNA, was screened by luminometry for luciferase activity using a Luciferase Assay System Kit (Promega, Madison, WI) on a microplate reader (BMG Labtech), as previously described (Shieh et al., Journal of Pediatric Surgery 2017; Tracy et al., Stem Cells and Development 29, 755-760, 2020; Lazow et al., Journal of Pediatric Surgery, 56, 1233-1236, 2021). Briefly, 100 μL of an assay solution containing luciferin and ATP was added to 20 μL of each sample's supernatant and samples were run in duplicates. Luminescence was measured in relative light units (RLU). To account for background noise, the luminescence of 3 samples containing lysis buffer only (blank) was measured and the mean plus 2 standard deviations of the blank was subtracted from each sample's raw RLU value. Total RLU in the entire lysed sample was then calculated, normalized by initial sample's weight or volume and expressed as RLU/mg or RLU/μL.

### Statistical Analysis

Fetal survival was compared between groups using the Fisher exact test. Luminescence data were compared by the nonparametric Mann-Whitney U-test. Statistical significance was defined by a two-tailed Bonferroni-adjusted p<0.05.

### Results

It was determined that the encapsulation rate of the firefly luciferase mRNA by lipopolyplex was 97.5%. The overall fetal survival at all time points was 87.5% (105/120), with no significant differences between the mRNA and control groups (p>0.28 for all pairwise comparisons). Survival of fetuses receiving mRNA lipopolyplexes was not significantly different cross time points (p>0.17 in pairwise comparisons), except between E20 (74%, 23/31) and E21 (100%, 23/23) (p=0.015). Maternal survival was 100%.

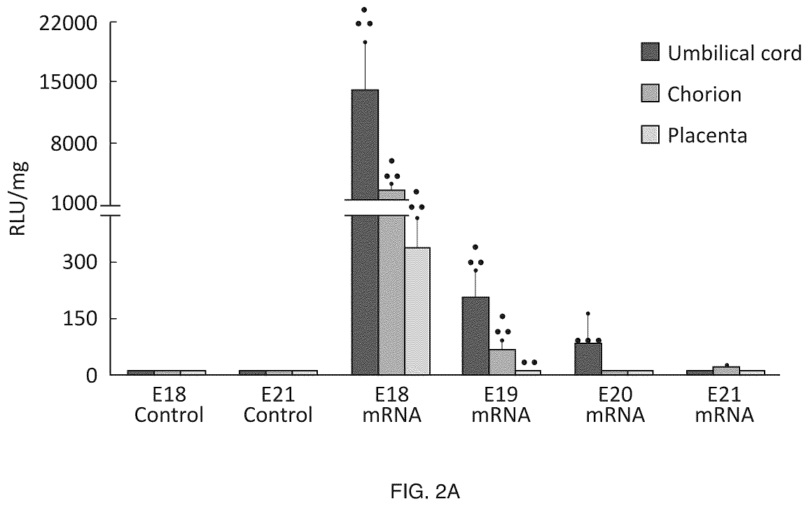

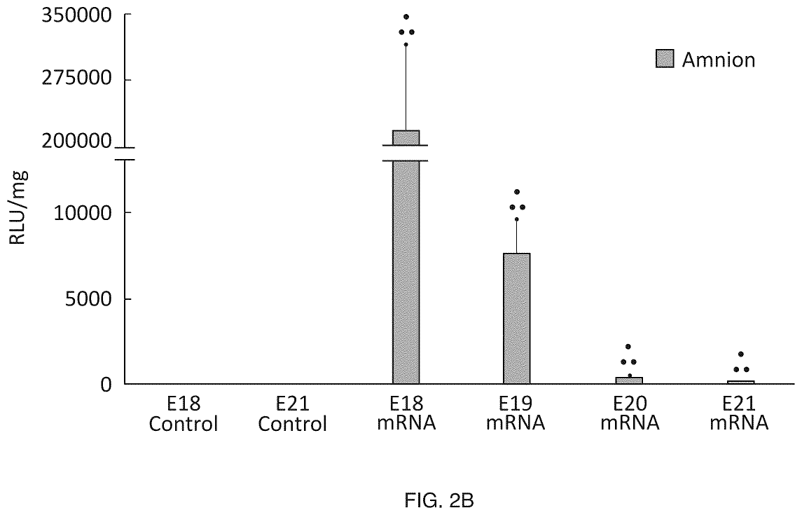

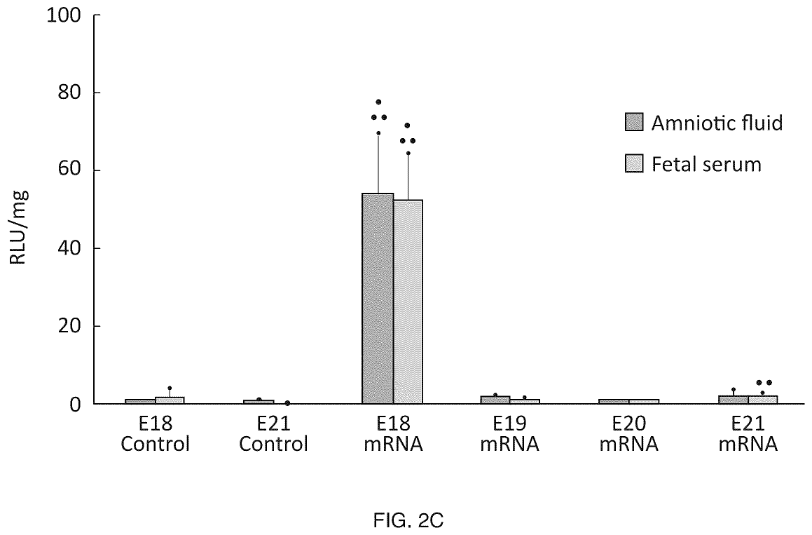

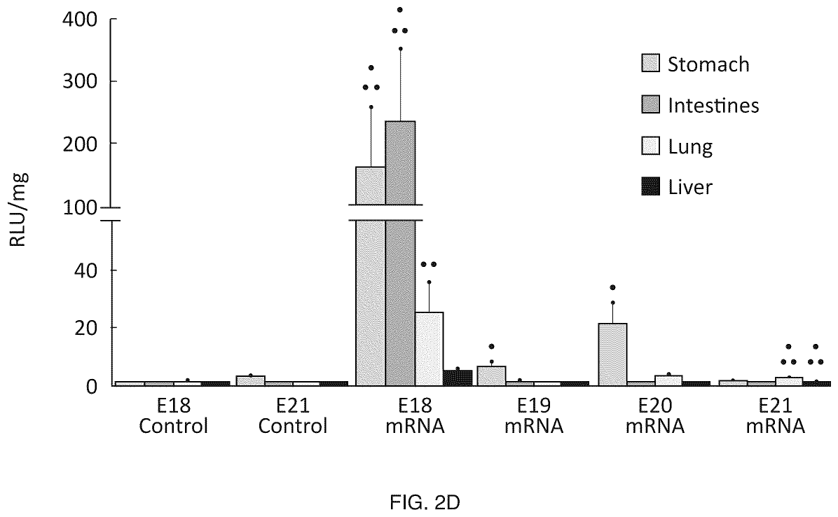

When controlled by the lipopolyplex without mRNA, luciferase protein activity was detected on E18 and E19 at the following fetal annexes: amnion, chorion, placenta and umbilical cord (FIGS. 2A and 2B). The highest peak was observed in the amnion on E18 (216150±67607.8 RLU/mg). Luciferase activity was detected in amniotic fluid only on E18 (FIG. 2C). Fetal incorporation of the luciferase mRNA was observed in the serum, stomach, intestines, lung, liver, all with peaks also on E18 (FIGS. 2C and 2D). No significant difference in luminescence between the two groups was observed at any time point in the fetal heart, brain, spleen and bone marrow (p=0.116-0.954 in all pairwise). Positive luminescence was detected in only one maternal serum sample, on E19 (253±126 RLU/μL).

While not intending to be bound by theory, the chronology of findings on the gestational membranes, umbilical cord, and placenta point to hematogenous routing through these structures as major components of the pharmacokinetics of transamniotic nucleic acid therapy (TRANAT), likely for the first time. At certain sites such as the liver, it is possible that both the intraluminal and hematogenous trafficking are concomitantly at play. The timeline of these results is also compatible with both passive and active transport, though the methods were not designed to discern whether either of these two scenarios predominates. The design was also not aimed at comparing the numerous different components available for mRNA encapsulation, but rather at testing the hypothesis that hematogenous routing might be present.

In light of the paucity of previous data on fetal mRNA administration, researchers elected to use a healthy model so as to establish baseline data for eventual further analyses in pathological states. It is possible that the diversity of such states, along with the diversity of the composition of encapsulation products, not to mention of the mRNA itself, can impact the distribution and cellular incorporation of the mRNA, including the duration of protein production by the host cells. The above examples include the firefly luciferase as the mRNA due to it being a highly sensitive tool for screening from minute samples in vivo. Although of course this protein is not produced by any mammal, a control group without mRNA was still included to be screened by luminometry, in order to minimize, if not eliminate, the possibility of false positives. In the interest of methodological consistency, all samples were processed comparably. The possibility cannot be ruled out that the homogenization process used may have been too aggressive for certain samples such as bone marrow, possibly leading to false negatives. Further, one notorious limitation of the luciferase assay is the fact that hemoglobin may interfere with it. This could have resulted in underestimation of the true amount of mRNA incorporation in organs with rich blood supply such as the bone marrow and spleen. Interestingly, luciferase production was detected in amniotic fluid samples on E18, suggesting mRNA incorporation and functionality by live cells present in the fluid. Given the previous demonstration of fetal (and maternal) hematogenous trafficking of amniotic fluid-derived mesenchymal and hematopoietic stem cells, the possibility cannot be ruled out that one or both these cell types, known to be present in the amniotic fluid, may have contributed to the evidences of hematogenous routing of the mRNA observed.

Despite the sensitivity and adequacy of firefly luciferase for the screenings performed, researchers could not obtain and process specimens from all sites analyzed (and others) at all time points, due to size restraints. It is possible that mRNA could have been present at such sites at earlier time points, yet simply not detected given that early peaks of luciferase activity on E18 were observed at many sites. It is also possible that mRNA encoding for a protein other than a completely alien one such as luciferase could have had a different pattern and timing of host cell incorporation. Of note, besides lung and liver, luciferase activity was present in the fetal serum at the longest time-point evaluated, i.e. 96 hours after the intra-amniotic injection.

TransIT is a self-assembling mRNA delivery system in which negatively charged mRNA spontaneously interacts with cationic lipids and polymers (To et al., Expert Opinion on Drug Discovery 16, 1307-1313, 2021). This explains the very high encapsulation rate measured, and also contributed to the stability of the lipopolyplex. Other systems could be considered for TRANAT. These results support the consideration of this novel strategy for pulmonary, gastrointestinal, hepatic, and serum/blood targets for prenatal mRNA therapy.

### Example 2: Transamniotic Delivery of Surfactant Protein B (SPB) mRNA: a Potential Novel Strategy for the Perinatal Management of Congenital SPB Deficiencies and Surfactant Replacement Therapy

Experiments were undertaken to determine whether exogenous encapsulated mRNA codifying for surfactant protein B (SPB) could be incorporated and translated by the fetal lung after transamniotic administration, as a prelude to the development of possible novel therapies for diverse pathological states that could benefit from SPB replacement or replenishment.

mRNA formulation and encapsulation A hSPB mRNA sequence was custom-ordered (NCBI: NM_000542.5; Ribo Pro, The Netherlands). That sequence was further modified to enhance translation efficiency, mRNA stability and to decrease immunogenicity by adding Cap 1 to 5′-end, 150nt PolyA-tail to 3′-end and by sequence optimization with proprietary nucleotide modification (Ribo Pro). To further amplify the mRNA stability and enhance delivery in vivo, the hSPB mRNA was encapsulated into self-assembling nanoparticles using the TransIT-mRNA transfection kit (Mirus Bio, Madison, WI), which consists of a lipid-and synthetic cationic polymer-based composite (a so-called lipopolyplex) with two components, namely a TransIT Reagent proper and a mRNA Boost Reagent. In accordance with the manufacturer's instructions, 1 μg of mRNA (1 μg/μL stock) was suspended in 45 μL of phosphate-buffered saline (PBS). After that, 2 μL of mRNA Boost Reagent and 2 μL of TransIT Reagent were added to the mixture. The final solution was pipetted gently and incubated for 5-10 minutes at room temperature prior to injection in vivo, so as to allow for mRNA lipopolyplex formation.

Encapsulation efficiency of mRNA into the lipopolyplexes was measured using Quant-iT RiboGreen assay (Thermo Fisher Scientific, Waltham, MA) as previously described (Jiang et al., Biomaterials 176, 122-130, 2018). Briefly, samples of naked mRNA in PBS (1 μg/50 μL stock) were plated along with mRNA encapsulated using TransIT into lipopolyplexes (1 μg/50 μL stock) and mRNA standards in a 96-well plate. Fluorescent RiboGreen reagent (Thermo Fisher Scientific, Waltham, MA) was then added and the resulting fluorescence intensity was measured on a microplate reader (BMG Labtech, Cary, NC) at 490 nm and 520 nm excitation and emission, respectively, in order to quantify the amount of free mRNA in solution. Encapsulation efficiency was calculated by subtracting the amount of free mRNA from the total originally used to generate the mRNA lipopolyplexes and expressed as a percentage.

Intra-amniotic injections

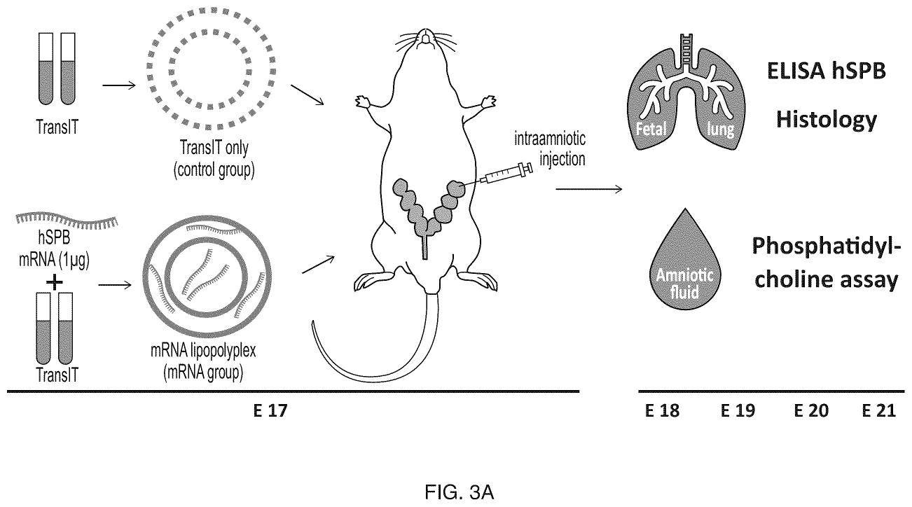

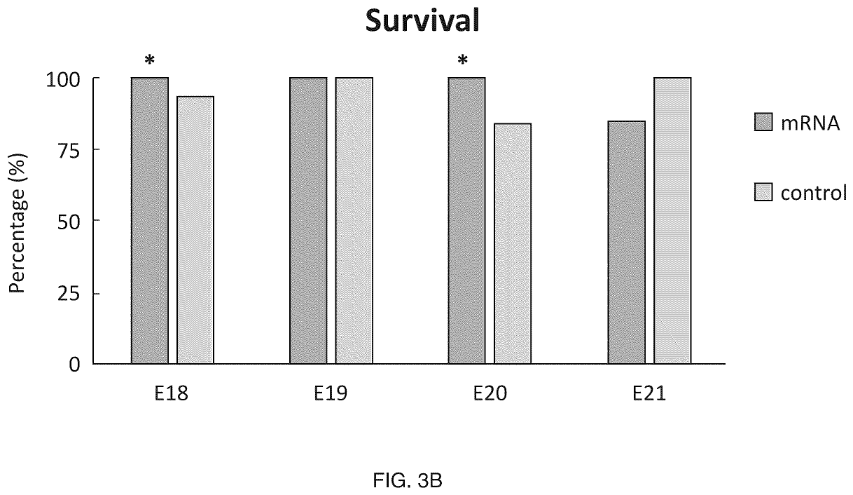

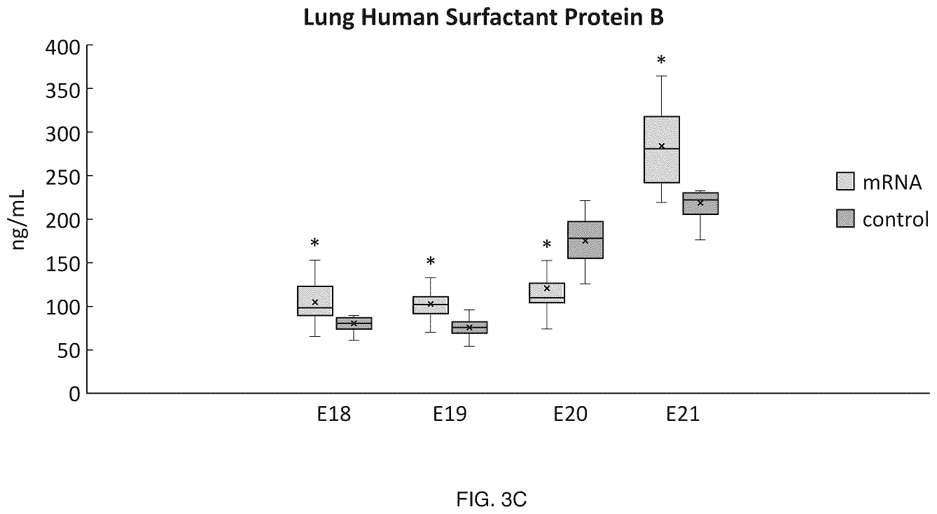

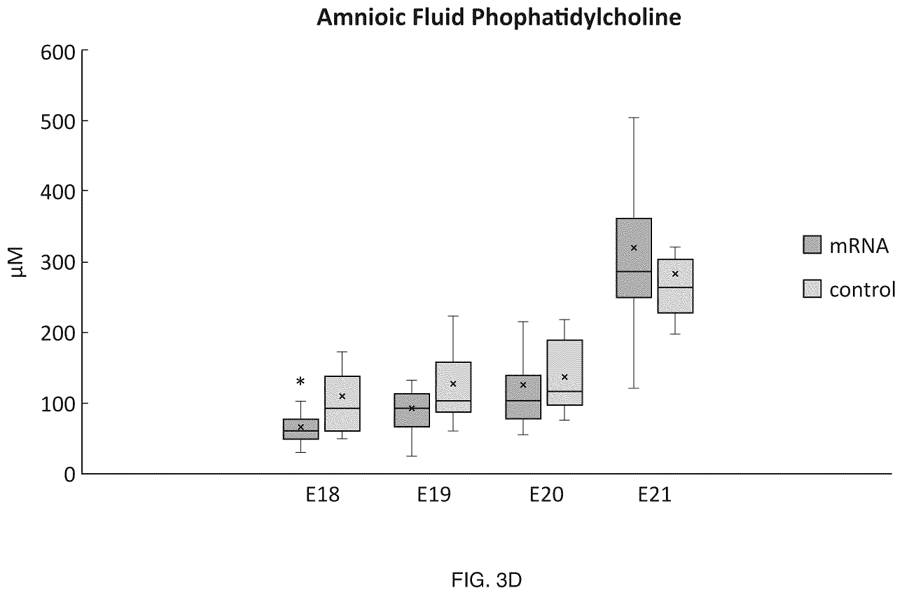

An overview of the experimental design is shown in FIG. 3A. Twelve Sprague Dawley dams (Charles River Laboratories, Wilmington, MA) underwent surgical intra-amniotic injections on gestational day 17 (E17, term=E21-22) as previously described (deidentified). Briefly, animals were anesthetized and maintained on 2-4% inhaled isoflurane (Patterson Veterinary, Greeley, CO) in 100% oxygen. A midline laparotomy was performed exposing the bicornuate uterus. The amniotic cavities of all viable fetuses (n=149) received volume-matched (50 μL) injections of a solution in PBS containing either the hSPB mRNA lipopolyplex (mRNA group; n=99), or the lipopolyplex composite without any mRNA (control group; n=50) using a 33G non-coring needle on a 100 μL syringe (both from Hamilton Company, Reno, NV). Injections were performed under direct vision with care to avoid injury to the fetus, placenta, or umbilical cord. The incision was closed in two layers and powdered metronidazole (Unichem Pharmaceuticals, Hasbrouck Heights, NJ) was applied to the wound. Sustained release buprenorphine (Fidelis Pharmaceuticals, North Brunswick, NJ) was administered for post-operative analgesia.

### Specimen Procurement

Dams from both groups were euthanized by CO2 chamber at daily time points from E18 to E21 (term). The laparotomy was reopened, the uterus was eviscerated and each fetus was removed en caul. Fetal lungs were then procured, along with amniotic fluid samples when there was enough of it to be aspirated. All of the left lung and most of the right lung were immediately frozen for enzyme linked immunosorbent assay (ELISA) analyses. A small sample of the right lung was fixed in 10% formalin for histology. Maternal blood was obtained via direct intra-cardiac puncture, then centrifuged at 2,000rcf for 8 min. to allow for separation of the serum, which was then obtained and immediately frozen. All sample freezing was by rapid dry ice-ethanol baths followed by storage at −80° C. until further processing.

hSPB ELISA

Fetal lung samples were standardized by weight and homogenized in PBS with stainless-steel beads (Next Advance, Averill Park, NY) in an automated tissue homogenizer (Next Advance) for 5 min. Lung homogenates were centrifuged at 10,000rcf for 5 min, and the supernatant was then used at a 1:20 dilution for hSPB detection using a commercially available hSPB ELISA kit (MyBioSource, San Diego, CA) according to manufacturer's instructions. Maternal serum samples were standardized by volume and used at 1:20 dilution for the same ELISA. The final hSPB concentration was calculated using a standard curve and expressed in ng/mL. All samples and standards were run in duplicates.

### Phosphatidylcholine Assay

A Phosphatidylcholine (PC) fluorescence assay kit (Abcam, Waltham, MA) was used to measure PC levels in the amniotic fluid as a surrogate for total surfactant production. Amniotic fluid samples were diluted at 1:25 using the proprietary assay kit buffer. Diluted samples and standards were incubated with the OxiRed Probe (Abcam) supplemented with a PC hydrolysis enzyme and PC development mix and were run in duplicates according to the manufacturer's instructions. Fluorescence intensity was measured on a microplate reader (BMG Labtech, Cary, NC) at 485 nm and 520 nm excitation and emission, respectively. Final amniotic fluid PC levels were calculated using a standard curve and expressed in μM.

### Statistical Analyses

Fetal survival between the groups was calculated using Generalized Estimating Equations (GEE) regression modeling to account for nesting of fetuses within dams. ELISA and PC data were compared by the Wilcoxon rank sum test. Statistical significance was defined as p<0.05. All data were presented as median with interquartile range (IQR) or as absolute numbers (for fetal survival analysis).

### Results

The encapsulation efficiency of the hSPB into the lipopolyplex was 96.7%. The overall fetal survival at all time points was 95% (141/149). There was a significantly higher survival in the mRNA group compared to controls at E18 (100% vs. 85.7%) and E20 (100% vs. 83.3%) (both p<0.001; FIG. 3B). There was no maternal mortality.

Fetal hSPB Production

The ELISA for hSPB protein was positive in the samples from the control group, suggesting some degree of human-rat homology for SPB. When controlled by mRNA-free injections, the hSPB protein was present in the fetal lungs of the mRNA group at E18, 19, and E21 (p=0.002 to <0.001; FIG. 3C). Lung levels of hSPB showed statistically significant increases over time in the mRNA group, with the highest levels at term (median 281.8 with IQR (242.4, 318.8); pairwise comparisons p=0.026 to <0.001). Both groups showed presence of hSPB in maternal serum samples, however no statistical comparisons between them could be performed due to the inadequate number of maternal samples for such analysis.

### Surfactant Production

Amniotic fluid PC levels trended higher in the mRNA group compared to control at term (respectively 319±30.2 vs. 281±29.3 ng/ml; FIG. 3D), however this did not reach significance in this healthy model (p=0.33).

Pulmonary surfactant is a complex lipoprotein mixture produced by the lungs to reduce surface tension at the air-liquid interface within the alveoli and allow for adequate gas exchange (Daniels et al., News Physiol Sci 18, 151-157, 2003). The protein portion of that surfactant mixture has 4 components-SPA, SPB, SPC and SPD—with the hydrophobic SPB holding the most significant role as it stabilizes the mixture and enhances its function by facilitating lipid absorption to the surface of the alveoli thus creating an active surface monolayer (Walther et al., Neonatology 91, 4, 303-310, 2007; Whitsett et al., Pediatr Res 20, 5, 460-470, 1986). The importance of SPB in surfactant function is further reinforced by the fact that mutations in the SPB gene can cause severe neonatal respiratory disease (Nogee LM, Annu Rev Physiol 66, 601-623, 2004). A prime example is congenital SPB deficiency, which is a monogenic autosomal recessive disease that leads to neonatal respiratory failure despite exogenous surfactant administration. The only current treatment option for it is lung transplantation in the first few months of life (Barnett et al., Exp Biol Med (Maywood) 242, 13, 1345-1354, 2017; Hamvas et al., J Pediatr, 125, 3, 356-361, 1994; Palomar et al, J Pediatr 149, 4, 548-553, 2006).

The much more common respiratory distress syndrome (RDS) of prematurity is directly linked to surfactant deficiency. Besides being the foremost cause of neonatal mortality, it is also associated with significant morbidity and chronic pulmonary diseases (Ng et al., Paediatr Child Health, 26, 1, 35-49, 2021; Polin et al., Pediatrics 133, 1, 156-163, 2014). Current management of neonatal RDS includes surfactant replacement based on either animal or synthetic products, each consisting of various ratios and mixtures of lipids and proteins (Polin et al., Pediatrics 133, 1, 156-163, 2014). Animal derived surfactants contain animal SPB and SPC while some of the synthetic products contain synthetic peptide functioning as a SPB analogs (Polin et al., Pediatrics 133, 1, 156-163, 2014). None of the currently available SPs is fully equal in composition and function to human SPs (Polin et al., Pediatrics 133, 1, 156-163, 2014; Hentschel et al., Pediatr Res 88, 2, 176-183, 2020). Despite the demonstrable benefits of current postnatal surfactant replacement therapies, there are still significant challenges to be overcome to further improve outcomes (Polin et al., Pediatrics 133, 1, 156-163, 2014; Hentschel et al., Pediatr Res 88, 2, 176-183, 2020; Battarbee et al., Am J Obstet Gynecol MFM, 2, 1, 100077, 2020, Joo et al, Neonatal Medicine, 29, 1, 46-54; Owen et al., Lancet 389, 10079, 1649-1659, 2017). Chief among these challenges is the creation of a stable surfactant product that would resemble human surfactant in both lipid and protein composition (Hentschel et al., Pediatr Res 88, 2, 176-183, 2020). Additionally, the administration of surfactant often requires either intubation or tracheal catheterization, as well as multiple dosages (Joo et al, Naonatal Medicine, 29, 1, 46-54).

Under normal conditions, SPB mRNA undergoes a series of translational steps within type II pneumocytes. First, it is translated to preproprotein (preproSPB), then to proprotein (proSPB), and then eventually modified into fully functional SPB (Nogee et al., Annu Rev Physiol 66, 601-623, 2004). This complex process carried by cell's endogenous machinery allows for natural post-translational modifications of exogenous mRNA and improved efficiency of the final protein product. Besides the possibility of being of human origin, this type of enhancement is another benefit of mRNA-based therapies for protein replacement, including SPB.

In this Example, it was shown that the minimally invasive transamniotic route is a viable alternative for the delivery of human SP to fetal lungs. Although the ultimate goal would be to develop a novel therapy for diseases such as congenital SPB deficiency and/or prematurity itself, the decision was made to start with a healthy model so as to establish baseline parameters of mRNA incorporation and translation by the host. The timeline of hSPB production starting as early as within 24 hours following intra-amniotic injection of the mRNA, along with the increasing hSPB levels for four days up to term, were all encouraging results, compatible with eventual clinical relevance. For example, such timeline would be suitable to the relatively acute setting of unanticipated preterm labor.

Of note in the present Example, control rat fetus showed presence of hSPB on ELISA. This is not exactly surprising in that surfactant proteins are known to commonly encompass interspecies homology. For example, up to approximately 80% of SPB's amino acid sequences are shared between humans and rats (Haagsman et al., Comp Biochem Physiol A Mol Integr Physiol 129, 1, 91-108, 2001). This was indeed why a control group receiving no hSPB mRNA was included in the experimental design at each time point.

Significantly improved survival was observed in the mRNA group compared to controls at E18 (100% vs. 85.7%) and E20 (100% vs. 83.3%) (both p<0.001). When controlled by mRNA-free injections, hSPB protein was detected in the mRNA group's lungs at E18, 19, and term (p=0.002 to <0.001, FIG. 3C). Amniotic fluid phosphatidylcholine levels were increased compared to control at term (319±30.2 vs. 281±29.3 ng/ml; FIG. 3D), however, these levels did not reach significance in this series (p=0.33).

Notwithstanding the limitations of this study, it has shown that exogenous encapsulated mRNA encoding for hSPB protein can be incorporated and translated by fetal lung cells after simple intra-amniotic injection in a healthy rat model. Transamniotic mRNA delivery could become a novel strategy for perinatal surfactant protein replacement or replenishment therapies.

### Example 3: Transamniotic Delivery of Cystic Fibrosis (CF) Transmembrane Conductance Regulator (CFTR) mRNA: a Strategy for the Perinatal Management of CF-Associated Meconium Ileus

Mutations of the cystic fibrosis transmembrane conductance regulator (CFTR), a cell membrane protein serving as chloride/anion channel in numerous epithelial cells, constitute the central component of the pathophysiology of cystic fibrosis (CF) and chief cause of neonatal meconium ileus associated with this disease. The gastrointestinal tract is commonly affected and as early as in the perinatal period, so that newborns with CF may suffer from meconium ileus as a result, followed by respiratory complications not long thereafter (Kelly et al., Dig Dis Sci 60, 1903-1913, 2015; Galante et al., Neoreviews 20, e12-e24, 2019; Sathe et al., J Cyst Fibros 16 Suppl. 2, S32-S39, 2017) The diagnosis of CF is often made early in pregnancy via genetic screening. In addition, certain fetal ultrasound findings may be predictive of meconium ileus after birth (Scotet et al., Am J Obstet Gynecol 203, 592 e1-6, 2010). Postnatal exogenous mRNA-based therapies for CF have been widely studied experimentally, having already reached clinical trials as inhalation therapies for its respiratory complications. Researchers sought to determine whether transamniotic nucleic acid therapy (TRANAT) could be a viable alternative for the administration of CFTR mRNA to the fetal bowel as a potential strategy for the perinatal management of meconium ileus associated with CF.

mRNA formulation and encapsulation A human cystic fibrosis transmembrane conductance regulator (hCFTR) mRNA sequence was custom-made (NCBI: NM_000492; Ribo Pro, The Netherlands) including the following modifications: Cap1 to deimmunize the sequence, 150nt PolyA-tail, and proprietary sequence optimization to yield increased translation into the final protein product. Just prior to injection in vivo, the custom hCFTR mRNA was encapsulated into semi-synthetic lipolyplex particles made of a self-assembling composite lipid and cationic polymer (TransIT; Mirus Bio, Madison, WI) consisting of a reagent proper and an mRNA boost reagent, using the TransIT-mRNA transfection kit (Mirus Bio) in accordance with the manufacturer's instructions. Briefly, 1 μg of mRNA (1 μg/μL stock) was suspended in 45 μL of phosphate-buffered saline (PBS), then 2 μL of each of the kit's two components (reagent and boost reagent) were added, followed by 5 minutes of incubation at room temperature to allow for encapsulation. Intra-amniotic injections were performed as described below.

To evaluate the efficiency of mRNA encapsulation into the lipopolyplexes, the Quant-iT RiboGreen assay (Thermo Fisher Scientific, Waltham, MA) was utilized as previously described [9]. Briefly, known amounts of encapsulated mRNA into lipopolyplex (1 μg/50 μL stock) were mixed with fluorescent RiboGreen reagent (Thermo Fisher Scientific). The amount of free-floating mRNA in that solution was then measured on a microplate reader (BMG Labtech, Cary, NC) by fluorescent detection at 490 nm and 520 nm excitation and emission, respectively. Encapsulation efficiency was determined by subtracting the amount of free mRNA in the lipopolyplex solution (lipopolyplex mRNA) from the total mRNA originally used (naked mRNA) and expressed as a percentage. The same procedure was performed in parallel using naked mRNA in PBS (1 μg/50 μL stock) instead of encapsulated mRNA, as positive controls.

### Intra-Amniotic Injections

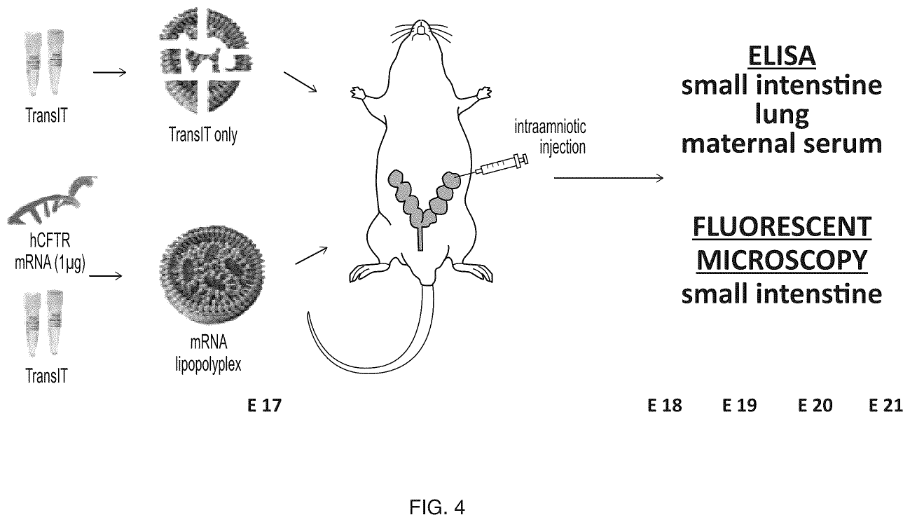

The overall experimental design is shown in FIG. 4. Nine time-dated pregnant Sprague Dawley dams (Charles River Laboratories, Wilmington, MA) underwent surgery for direct intra-amniotic injections on gestational day 17 (E17, term=E21-22) as previously described (Jiang et al., Biomaterials 176, 122-130, 2018). In brief, dams were sedated with 2-4% inhaled isoflurane (Patterson Veterinary, Greeley, CO) in 100% oxygen. Following a midline laparotomy to expose bicornuate uterus, all fetuses (n=109) received volume-matched intra-amniotic injections (50 μL) of either a suspension of a hCFTR mRNA lipopolyplex (mRNA group; n=98), or a suspension of the encapsulation composite but free of mRNA (control group; n=11) using a 33G non-coring needle on a 100 μL syringe (both from Hamilton Company, Reno, NV). Injections were performed under direct vision with care to avoid injury to the fetus, placenta, or umbilical cord. The incision was closed in two layers and powdered metronidazole (Unichem Pharmaceuticals, Hasbrouck Heights, NJ) was applied to the wound. Post-operative analgesia was by extended-release buprenorphine (Fidelis Pharmaceuticals, North Brunswick, NJ).

### Specimen Procurement

Dams were euthanized by CO2 chamber at daily time points following the intra-amniotic injections, from E18 to E21 (term) for the mRNA group and at E21 only for the control group. Samples from fetal small intestine and lung were procured as previously described (Lazow et al., J Petiatr Surg 56, 1233-1236, 2021). Small intestine samples from E19, E20 and E21 fetuses were divided into 2 halves, with one being immediately frozen for enzyme linked immunosorbent assay (ELISA) analyses while the other was fixed in 10% formalin for histology. Intestinal samples from E18 animals were not divided due to their very small size and were only frozen for ELISA. Maternal blood was obtained via direct intra-cardiac puncture and centrifuged at 2000 rcf for 8 minutes for serum separation and storage. All sample freezing was by rapid dry ice-ethanol baths followed by storage at −80° C. until further processing.

### ELISA

Fetal small intestine and lung samples were standardized by weight and homogenized. Maternal serum samples were standardized by volume. All samples were screened for the presence of the hCFTR protein using a commercially available ELISA kit (Biorbyt, Cambridge, United Kingdom) in accordance with the manufacturer's instructions. The final hCFTR protein concentration in ng/mL was calculated after all samples and standards were run in duplicate and averaged.

### Immunofluorescence

Immunofluorescent staining for hCFTR was performed on subsets of formalin fixed fetal small intestines to assess the topography of cellular distribution of the protein within the enterocytes. Three samples from the mRNA group, each from a different time-point (E19-E21), and two samples from the control group at E21 were randomly selected. Samples were paraffin embedded and sectioned. Paraffin section slides were then deparaffinized and rehydrated following 15 minutes in xylene, twice. Specifically, 5, 5, and 5 minutes in 100%, 100%, and 75% ethanol, respectively, and 5 minutes in PBS at room temperature repeated three times. Slides were heated up to 110° C. for 15 minutes, then cooled down to room temperature for 30 minutes and washed with Tris-buffered saline/Tris-buffered saline with 0.1% Tween 20 detergent (TBS/TBST) for 5 minutes, three times. Slides were then blocked with normal goat serum and incubated with primary antibodies (hCFTR C-terminus antibody; Biotechne R&D Systems, Minneapolis, MN) overnight at 4° C. Following repeated TBS/TBST wash for 5 minutes three times, slides were incubated with secondary antibodies (Goat anti-Mouse IgG, Alexa Fluor; Thermo Fisher Scientific) for 1 hour at room temperature. Finally, slides were stained with nuclear 4′,6-diamidino-2-phenylindole (DAPI). Immunolabeled sections were examined on a Zeiss LSM 880 Confocal Microscope (Zeiss, Oberkochen, Germany) under oil lenses. Representative images were taken at 63× magnification.

### Statistical Analyses

Fetal survival comparisons between the groups was by Fisher exact test. ELISA data were compared by nonparametric Wilcoxon rank sum test. Statistical significance was defined by a Bonferroni-adjusted p<0.05.

### Results

The encapsulation rate of the hCFTR mRNA into the lipopolyplex was 99.5%. Overall fetal survival was 85.3% (93/109), with no significant differences between the groups or time-points (p>0.067 for all). There was no maternal mortality.

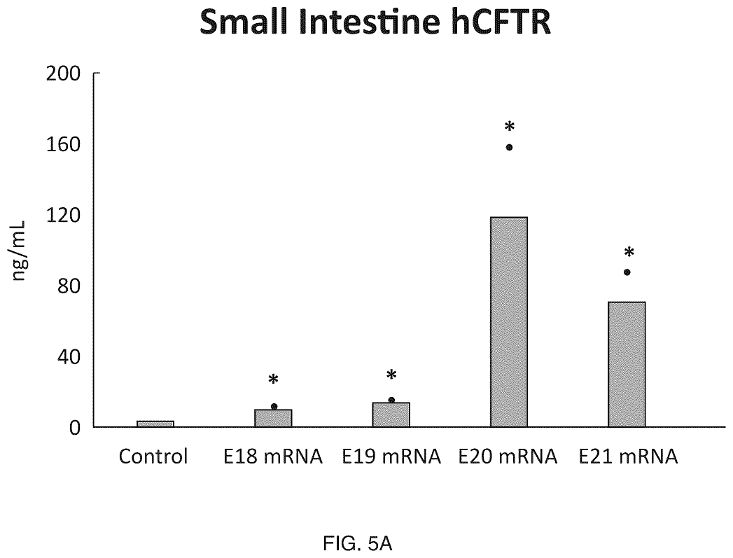

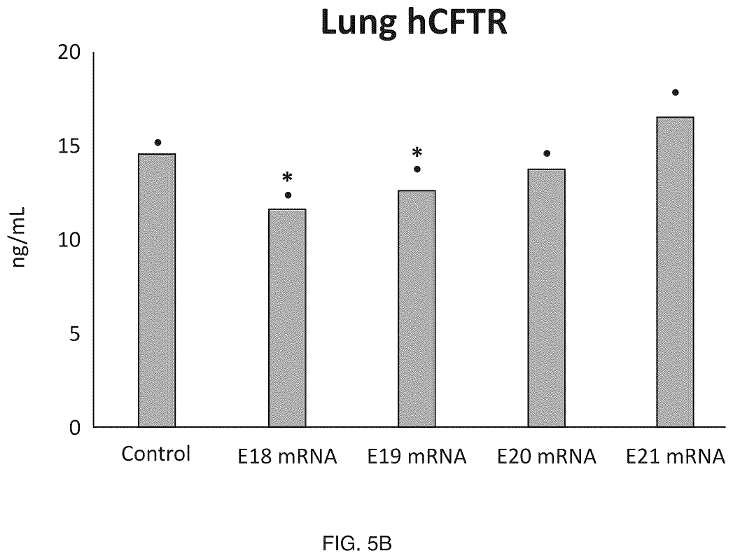

When controlled by mRNA-free injections, hCFTR protein was detected in the fetal small intestine of the mRNA group at all time points (p<0.001 for all, FIG. 5A). The amount of intestinal hCFTR tended to increase over three days after intra-amniotic mRNA delivery (average at E20=118±40 ng/mL), with persistently high levels at term (average at E21=70±17 ng/mL). Interestingly, the hCFTR protein was detected in the fetal lung in both the mRNA and control groups with even significantly decreased amounts at E18-19 in the mRNA group vs. control (p<0.012 for both; FIG. 5B), suggesting the possibility of some degree of interspecies homology at least at this anatomical site. When controlled by mRNA-free injections, no hCFTR was detected in any maternal serum sample (p>0.02 for all).

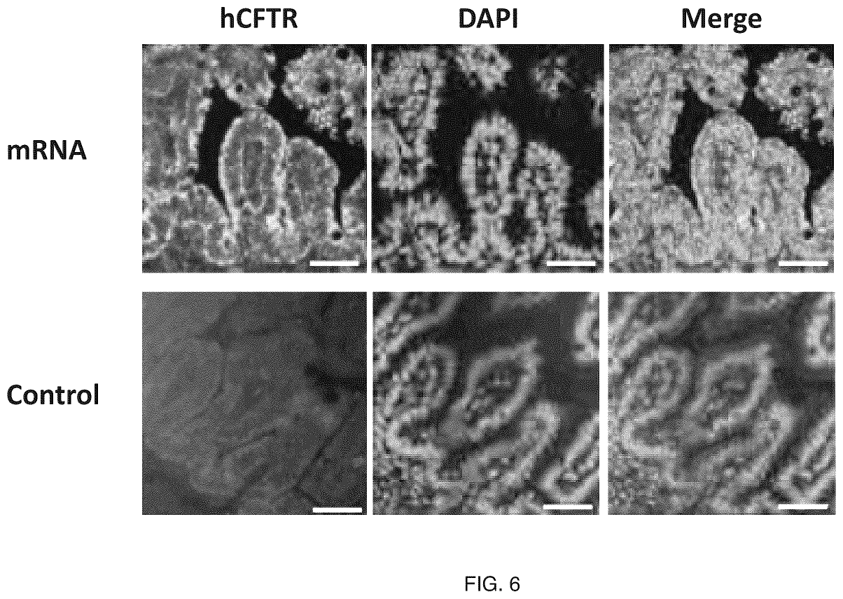

Representative immunofluorescent images showed strong, concentrated fluorescent signal at the apical membranes of enterocytes in the small intestine in the mRNA group, in accordance with typical apical hCFTR localization (FIG. 6). No hCFTR signal was observed in the control group.

This data showed consistent and enduring cellular incorporation of the hCFTR mRNA in the fetal intestine and its local translation to the hCFTR protein following transamniotic delivery. Given the minimally invasive nature of a simple intra-amniotic injection and the fact that the fetus is frequently swallowing amniotic fluid, this form of fetal mRNA delivery may offer advantages over an eventual postnatal form of administration, which has yet to be proven viable for the intestine. It has been shown, however, that encapsulated hCFTR mRNA can be incorporated by and function in adult mice lung for 6-14 days following intravenous or intratracheal nebulized administration. Even if postnatal administration proves viable, an additional benefit of the fetal delivery is the presence of the protein already at birth, which could be more effective in preventing meconium ileus in that patient population.

Clinically relevant CFTR mutation patterns and consequences range from transcription disruptions leading to misfolded CFTR not reaching apical cell membranes to properly positioned chloride channel/CFTR protein that are severely malfunctioning so as to not allow for adequate ion transport. The fluorescence microscopy data showed that a majority of the hCFTR protein was localized in the expected normal apical topography within the enterocytes. Fluorescence microscopy data showed that a majority of the hCFTR protein was localized in the expected normal apical topography within the enterocytes. This is an encouraging finding, to be considered under the light that it has been previously shown in a transgenic porcine model of cystic fibrosis (CF) associated meconium ileus, that as little as 20% of CFTR mRNA function in the intestine is sufficient to prevent meconium ileus.

It was intriguing to detect hCFTR protein in the fetal lungs from the control group at comparable levels to those detected in lungs from the mRNA group. While not intending to be bound by theory, one possible explanation could be that there is some degree of interspecies homology at this particular anatomical site. Another possible explanation has to do with the mRNA product that was used. The mRNA product was prepared via a proprietary sequence optimization technology developed by Ribo Pro that increases protein expression on average fivefold by removing toll-like receptors that activate sub-sequences that negatively impact protein expression.

These data show that exogenous mRNA encoding for hCFTR protein can be incorporated and translated by fetal small intestine cells after simple intra-amniotic injection in a healthy rat model. Transamniotic nucleic acid therapy (TRANAT) could become a novel strategy for the perinatal management of meconium ileus associated with cystic fibrosis.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for treating cystic fibrosis in a developing fetus, the method comprising: administering to the amniotic fluid surrounding a developing fetus an mRNA molecule encoding a cystic fibrosis transmembrane conductance regulator (CFTR) polypeptide, thereby treating cystic fibrosis in the developing fetus.

2. The method of claim 1, wherein the mRNA is translated in the lungs and/or intestines of the fetus to produce the CFTR polypeptide in lung cells of the fetus.

3. The method of claim 1, wherein the method comprises reducing the incidence of meconium ileus in the fetus or the child developed from the fetus.

4. A method for treating a disease or disorder associated with a surfactant protein B (SPB) deficiency in a developing fetus, the method comprising: administering to the amniotic fluid surrounding a developing fetus an mRNA molecule encoding an SPB polypeptide, thereby treating the disease or disorder associated with SPB deficiency in the developing fetus.

5. The method of claim 4, wherein the disease or disorder is prematurity or congenital surfactant protein B deficiency.

6. The method of claim 4, wherein the mRNA is translated in small bowel cells or lungs of the fetus to produce the SPB polypeptide in small bowel cells of the fetus or in lungs of the fetus, respectively.

7. The method of claim 4, wherein the method is associated with an increase in levels of phosphatidylcholine in the amniotic fluid.

8. The method of claim 1, wherein the mRNA is administered using a lipid nanoparticle or a lipopolyplex.

9. The method of claim 1, wherein the fetus is a human fetus.

10. The method of claim 1, wherein the mRNA is administered prior to 10 weeks of pregnancy.

11. The method of claim 1, wherein the mRNA is administered prior to 5 weeks of pregnancy.

12. The method of claim 1, wherein the mRNA is administered after 10 weeks of pregnancy.

13. The method of claim 1, wherein the administering is associated with the presence of and/or an increase in levels of the mRNA in a tissue of the fetus.

14. The method of claim 13, wherein the tissue comprises liver, stomach, intestines, pancreas, spleen, thymus, lymph nodes, brain, meninges, heart, blood vessels, lungs, airways, kidneys, ureters, urethra, ovaries, testicles, genitalia, skin, skin annexes, muscle, bone, cartilage, bone marrow, eyes, ears, mouth, nose, pharynx, larynx, vitreous body, cerebrospinal fluid, and peripheral blood.

15. The method of claim 14, wherein the tissue comprises liver, stomach, intestines, lungs, and/or blood.

16. The method of claim 4, wherein the administration is associated with the presence of and/or an increase in levels of the mRNA in amniotic fluid, amnion, chorion, umbilical cord, and/or placenta associated with the fetus.

17. A kit suitable for use in treating a disease or disorder, wherein the kit comprises the mRNA of claim 1.

18. A method for delivering one or more mRNA molecules to the bloodstream of a developing fetus, the method comprising: administering to the amniotic fluid surrounding the developing fetus an mRNA molecule encoding a polypeptide that has reduced expression and/or activity in the developing fetus relative to a healthy developing fetus.

19. The method of claim 18, wherein the mRNA is translated in an organ and/or tissue of the developing fetus, thereby treating the developing fetus for a disease or disorder associated with the polypeptide.

20. The method of claim 18, wherein the polypeptide is surfactant protein A, B, C, or D.
